(19) United States
(12) Patent Application Publication
(10) Pub. No.: US 2013/0131423 A1
(43) Pub. Date: May 23, 2013

(54) Methods to detect and treat diseases

(71) Applicants: Tianxin Wang, Burlingame, CA (US); Wenbin Ma, Frederick, MD (US); Yiwang Chen, Potomac, MD (US); Lei Liu, Potomac, MD (US)

(72) Inventors: Tianxin Wang, Burlingame, CA (US); Wenbin Ma, Frederick, MD (US); Yiwang Chen, Potomac, MD (US); Lei Liu, Potomac, MD (US)

(21) Appl. No.: 13/444,201

(22) Filed: Apr. 11, 2012

Related U.S. Application Data
(60) Provisional application No. 61/457,801, filed on Jun. 7, 2011.
(60) Provisional application No. 61/516,956, filed on Apr. 12, 2011.
(60) Provisional application No. 61/457,807, filed on Jun. 8, 2011.

(51) Int. Cl. A61N 5/02; A61M 1/36; C12N 5/0781; A61N 5/06

(52) U.S. Cl. CPC A61M 1/3621; C12N 5/0635
USPC 600/1; 435/2; 604/522; 607/88

(57) ABSTRACT

The current invention discloses methods to treat disease caused by virus infection, bacterial infection, parasites infection, autoimmune disease, disease caused by production of unwanted antibodies, sepsis as well as methods to treat cancer and methods for virus infection detection using blood purification method. The current invention provides a method to treat pathogen infection by inactivating the pathogens in the blood. During the treatment, blood is withdrawn from a patient and is separated into its plasma and cellular components. The plasma portion is treated with physical means such as UV radiation to inactivate the pathogens inside and then is returned to the patient. The current invention also provide a method to treat cancer especially to prevent tumor metastasis and tumor recurrence by removing and/or inactivating (e.g. killing) the circulating tumor cells (CTC) in the blood after removing the tumor or treating the tumor with therapeutical means.

### CROSS-REFERENCE TO RELATED APPLICATIONS

This application claims priority to U.S. Provisional Patent Application No. 61/457,807 filed on Jun. 8, 2011 and U.S. Provisional Patent Application No. 61/516,956 filed on Apr. 12, 2011. The entire disclosure of the prior application is considered to be part of the disclosure of the instant application and is hereby incorporated by reference.

### BACKGROUND OF THE INVENTION

1. Field of the Invention

The current invention relates to methods to treat disease caused by virus infection, bacterial infection and parasites infection as well as methods to treat cancer. The current invention also relates to methods to treat autoimmune disease, disease caused by production of unwanted antibodies.

2. Background Information

Extracorporeal therapy is a procedure in which blood is taken from a patient's circulation to have a process applied to it before it is returned to the circulation. All of the apparatus carrying the blood outside the body is termed the extracorporeal circuit. It includes hemodialysis, hemofiltration, plasmapheresis, apheresis and etc. Hemodialysis is a method for extracorporeal removing waste products such as creatinine and urea, as well as free water from the blood when the kidneys are in renal failure. Plasmapheresis is the removal, treatment, and return of (components of) blood plasma from blood circulation. The procedure is used to treat a variety of disorders, including those of the immune system, such as myasthenia gravis, lupus, and thrombotic thrombocytopenic purpura. Hemoperfusion (blood perfusion) is a medical process used to remove toxic or unwanted substances from a patient's blood. Typically the technique involves passing large volumes of blood over an adsorbent substance. The adsorbent substances most commonly used in hemoperfusion are resins and activated carbon. Hemoperfusion is an extracorporeal form of treatment because the blood is pumped through a device outside the patient's body. Its major uses include removing drugs or poisons from the blood in emergency situations, removing waste products from the blood in patients with renal failure, and as a supportive treatment for patients before and after liver transplantation. Apheresis is a medical technology in which the blood of a donor or patient is passed through an apparatus that separates out one particular constituent and returns the remainder to the circulation. Depending on the substance that is being removed, different processes are employed in apheresis.

### BRIEF DESCRIPTION OF THE DRAWINGS

FIG. 1 shows an example of the blood of a patient with virus infection passes through a plasma separator and is treated with pathogen inactivating means.

FIG. 2 shows a plasma separator filled with pathogen adsorbent for virus removal.

FIG. 3 shows a CTC (circulating tumor cells) removal cartridge.

FIG. 4 shows a CTC removal cartridge filled with CTC adsorbent.

FIG. 5 shows a CTC removal cartridge without CTC outlet.

FIG. 6 shows a CTC removal cartridge filled with CTC adsorbent without CTC outlet.

FIG. 7 shows filter based CTC removal devices without hollow fiber.

FIG. 8 shows three filters placed sequentially to remove CTC.

FIG. 9 shows an example of extracorporeally circulating blood CTC removal system.

The figures in the current inventions are for illustration purpose and may not accurately describe the size/relative size of each component.

### DESCRIPTION OF THE INVENTIONS AND THE PREFERRED EMBODIMENT

The first aspect of the current inventions disclose methods to treat autoimmune disease/diseases caused by the production of certain antibody (In the current inventions the “/” mark means either “and” or “or”). Many diseases are now related to autoimmune problem or the production of unwanted antibody which is harmful, e.g. diabetes, arthritis, allergy and etc. The method in the current invention to treat these problems involves two steps, in the first step; antibodies or specific antibody causing the disease is removed by blood purification procedure (e.g. hemopurification, plasmapheresis, blood perfusion, plasma exchange, immune absorption or blood dialysis). Hemopurifier and blood dialysis device are widely used for many disease such as kidney failure, drug poison. One can use either non-specific method to remove all the antibodies from blood (e.g. using protein A coated column, active carbon filter, membrane differential filtration, cryofiltration, plasmapheresis, plasma exchange) or specific method to selectively remove certain antibodies specific to certain antigen (e.g. using column coated with specific antigen, immuo adsorbent). The blood purification operations used in the current inventions can either be whole blood (both blood cells and plasma) purification or plasma purification by removing the blood cells before purification. These techniques are well known to the skilled in the art.

Examples of removing antibodies or certain specific antibody from blood can be found in many references; e.g. those described in: Extracorporeal removal of circulating immune complexes: from non-selective to patient-specific, Blood Purif 2000; 18:156-160; Antigen-specific apheresis of pathogenic autoantibodies from myasthenia gravis sera, Ann N Y Acad. Sci. 2008; 1132:291-9; and Selective removal of anti-acetylcholine receptor antibodies and IgG in vitro with an immunoadsorbent containing immobilized sulfathiazole, Artif Organs. 1990 October; 14 (5):334-41.

After the circulating target antibody is removed from the patient (in some embodiments this step can also remove the circulating immune cells that can selectively bind with the antigen for the target antibody if whole blood perfusion is used), a reagent that can selectively inactivate the cell (e.g. B cells) that involves the production of specific target antibody or that can selectively inactivate certain T cell targeting the specific disease causing antigen for the target antibody is given, e.g. the antigen-toxin conjugate such as hot suicide antigen or the like (e.g. antigen-cell inactivator conjugate or antigen-cell inhibitor conjugate, such as inhibitors or antisense molecule or siRNA that can inhibit the immune cell's normal function of producing antibodies but may not necessarily kill the cell) can be given (e.g. injected) to the patient. These antigen-toxin conjugate or the like will bind with B cell that express/produce specific target antibody binding with this antigen so the B cell or related immune cell will be inactivated or killed. They may also bind with the target antigen specific T cell therefore inactivates these T cells. Therefore the patient will not produce antibody targeting this antigen anymore and will not be reactive to this antigen. And the administration (e.g. injection) of hot suicide antigen or the like will not cause the generation of significant amount of antibody-antigen immune complex (since most of the antibodies for this antigen is removed in the previous step), which can precipitate in some organs and cause damage. After the hot suicide antigen or the like is given to the patient, a blood purification procedure can be performed to further remove the residual target antibody and the formed antibody—hot suicide antigen or the like immune complex. If desired, multiple dose of hot suicide antigen or the like can be given to the patient. Furthermore, excess hot suicide antigen or the like can be removed from the patient after the treatment using additional blood purification. These hot suicide antigens or the like can also bind with T cells that bind with them selectively and therefore inactivate these cells as well to reduce the autoimmune effect. Certain T cell can also selectively bind with the target antigen and generate immune response, killing/inactivating these T cells can also reduce the undesired immune effect, which are the cause of some diseases such as certain type of diabetes. Examples of the antigen can either be the whole antigen (e.g. protein) or part of it (e.g. epitope such as peptides) or peptide mimetic or small molecules that can bind with the antibodies, or other affinity molecules such as proteins, peptides or small molecules that can bind with the unique marker of the target cells surface that need to be inactivated. The affinity ligand (e.g. antibody) that can bind with these target immune cells can be used to couple with toxin/cell inhibitor/inactivator to be used instead. This method can selectively inactive the immune response to certain antigen without causing side effects produced by antibody-antigen immune complex, therefore it can also be used to treat other diseases caused by certain antibodies such as organ transfer, some bacterial, virus infection and etc. Many hot suicide antigens or the like has been reported and these reagents and procedures can be readily adopted for the current invention. For example, the publication in Scand J Immunol. 1985 November; 22(5): 489-94 described elimination of trinitrophenol-specific antibody response by antigen-toxin conjugates; the publication in The Journal of Immunology, Vol 131, 1983, Issue 4 1762-1764 described selective inhibition of anti-nucleoside-specific antibody production by nucleoside-ricin A conjugate; the publication in the Journal of experimental medicine. Volume 136, 1972, 305 described deletion of hapten-binding cells by a highly radioactive 1125 conjugate; the publication in Leuk Lymphoma. 2003 April; 44(4):681-9 described specific destruction of hybridoma cells by antigen-toxin conjugates demonstrates an efficient strategy for targeted drug therapy in leukemias of the B cell lineage; the publication in the Journal of Immunology, Vol 133, 1984, Issue 5 2549-2553 described specific killing of lymphocytes that cause experimental autoimmune myasthenia gravis by ricin toxin-acetylcholine receptor conjugates; the publication in Science 10 Jan. 1986: Vol. 231. no. 4734, pp. 148-150 described specific immunosuppression by immunotoxins containing daunomycin; the publication in Proc Natl Acad Sci USA. 1987 October; 84(20):7232-6 described antigen-specific drug-targeting used to manipulate an immune response in vivo; the publication in J Immunol. 1986 Nov. 15; 137(10):3135-9 described selective in vitro inhibition of an antibody response to purified acetylcholine receptor by using anti-idiotypic antibodies coupled to the A chain of ricin; the publication in J. Immunol. 1985; 135; 3062-3067 described selective in vitro inhibition of an antibody response to purified acetylcholine receptor by using antigen-ricin A chain immunotoxin. The B cell clonal toxins described in (WO/2001/032853; B cell clonal toxins and methods for using the same) and those used by Institute for Applied Biomedicine (e.g. Immudel-gp 120) are also this kind of certain B or T cell eliminating/inactivating agents that can be used in the current invention.

Examples of toxin/cell inhibitor/inactivator include but not limited to any agent that can kill the cell or inhibit the cell's normal or specific function (e.g. producing certain molecules such as protein (e.g. antibody), replication, differentiation, growth, developing into mature cell or other type of cell). They could be radioactive isotope, proteins, small molecules, siRNA, antisense molecules, enzymes and etc. Examples of them include NK cytotoxic factor, TNF such as TNF-α and TNF-β (LT), perforin, granzyme, cell apoptosis inducers, free radical generating agent, cell membrane damaging agent, toxic agent, chemotherapy agent, siRNA or antisense nucleic acid for the cell normal function, cytotoxic agent and etc. Sometimes they can be made to be in precursor type or inactive type and only become active after they bind with target cell or been taken by the target cell, e.g. the antigen-donomycin conjugate described above. Using affinity molecules coupled with cell damaging reagent is widely used in the treatment of tumor. One can readily adopt the method and principle of them for the current invention. If the cell-damaging reagent is effective only inside the cell, it normally involves a mechanism crossing the cell membrane such as endocytosis.

In some embodiments, patient having myasthenia gravis is first treated with blood purification method to remove the circulating antibodies against acetylcholine receptor in the blood. Varieties of blood purification techniques can be used such as those described in the above reference. One method is to use column immobilized with acetylcholine receptor protein to selectively remove the antibodies for it in the blood pass through. Next, antigen-toxin conjugates such as acetylcholine receptor-daunomycin or acetylcholine receptor-ricin A chain immunotoxin is given (e.g. injected) to the patient to selectively inactivate the acetylcholine receptor antibody producing immune cells. Examples of the antigen-toxin conjugate can be found in the above-cited reference. The amount of the drug injected can be determined experimentally. The suitable amount should have high target immune cell inactivating capability yet low side effect. In one example, patient having myasthenia gravis is first treated with blood purification method to remove the circulating antibodies against acetylcholine receptor in the extracorporeally circulating blood. The blood of a patient with myasthenia gravis passes through a plasma separator. The plasma part passes through a anti-acetylcholine receptor antibody removal column (e.g. a column filled with 50 g immune adsorbent carrying human AChR extracellular domains described in Ann N Y Acad. Sci. 2008; 1132:291-9) and then the treated plasma is combined with the blood cells from the plasma separator. The cleaned blood is sent back to the patient. The blood flow rate is 150 ml/min and the treatment continues for 2 h. Alternatively, total antibody can be removed non-selectively (e.g. using Immunosorba system from Fresenius Medical Care). Next one hour after the blood purification, the patient is injected with 1-125 labeled human AChR extracellular domain (described in Ann N Y Acad. Sci. 2008; 1132:291-9) at a single dose of 10 mCi. The ratio of iodine:human AChR extracellular domain is 0.9:1. Alternatively, the patient is injected with ricin toxin-human acetylcholine receptor conjugates at the dose of 0.1 ug/kg (prepared according to J. Immunol. 1984; 133; 2549-2553). Optionally the above blood purification can be used again to remove the residual circulating antibody. A C1q column can also be used to remove the formed circulating immune complex in the blood. If required, additional doses of the said 1-125 labeled human AChR or ricin toxin-human acetylcholine receptor conjugates can be given to the patient until the desired treatment efficacy is reached.

In another example, patient having diabetes is first treated with blood purification method to remove the circulating antibodies against diabetes antigen (e.g. GAD 65, IA-2, beta cell surface antigen, insulin receptor and insulin) in the blood. Varieties of blood purification techniques can be used such as those described above. For example, one method is to use column immobilized with insulin receptor protein/beta cell antigen to selectively remove the antibodies for them in the extracorporeally circulating blood passing through. Another example is to use none selective method such as active carbon absorption or protein A column/filter or membrane differential filtration to remove all the antibodies in the blood. Next, antigen-toxin conjugate such as insulin receptor-daunomycin or insulin receptor-ricin A chain immunotoxin or beta cell antigen-toxin conjugate is injected to the patient to selectively inactivate the insulin receptor antibody/beta cell antibody producing immune cells. Examples of the antigen-toxin conjugate can be made using the methods in the above-cited reference. The amount of the drug injected can be determined experimentally. The suitable amount should have high target immune cell inactivating capability yet low toxicity.

Yet in another example, patient having rheumatoid arthritis is first treated with blood purification method to remove the circulating antibodies against rheumatoid arthritis antigen in the extracorporeally circulating blood. Many rheumatoid arthritis antigens have been discovered such as Sa antigen, A47, GPI, HLA-DRB1-binding peptide and HA308-317 peptides. Varieties of blood purification techniques can be used such as those described above. For example, one method is to use column immobilized with these antigens to selectively remove the antibodies for them in the blood passing through. Another example is to use none selective method such as active carbon absorption or protein A column/filter or membrane differential filtration to remove all the antibodies in the blood. Next, antigen-toxin conjugate such as I-125-GPI, GPI-225 daunomycin, A47-ricin A chain immunotoxin are injected to the patient to selectively inactivate the specific immune cells. Examples of the antigen-toxin conjugate can be made using the methods in the above-cited reference. The amount of the drug injected can be determined experimentally. The suitable amount should have high target immune cell inactivating capability yet low side effect.

Similarly, this method can also be used to treat allergy once the antigen/antigens causing allergy are identified. First, all the antibodies in the blood or only the antibodies specific to the allergy antigen can be removed from blood as described above. Next, allergy causing antigen-toxin conjugate or multiple antigen-toxin conjugates or the like are injected to the blood to selectively inactivate these antibodies producing cells (e.g. certain B cell) and these antibodies specific immune cells (e.g. certain T cells).

Furthermore, the protein (e.g. antibody) that produced by T cell and B cell having affinity to these antigens can be identified. For example, these antibodies can be captured with affinity column via blood purification/filtration procedure described above. Next these antibodies or the like can be sequenced and the corresponding mRNA sequence can be determined. Affinity ligands (e.g. antibodies, small molecules, aptamers) that are specific to these antibodies or the like and can block the binding of these antibodies or the like to the antigens can be applied to the patient to treat the corresponding immune disease. Also, inactivating agents such as siRNA or antisense molecules targeting these mRNA can also be used to block the generation of these proteins by administrating them to the patient to treat the corresponding immune problem. One can isolate the protein and determine the mRNA for each patient and provide customized therapy. A database can also be generated to cover the most prevalent mRNA of these proteins for certain disease from many patient's sample and use most prevalent mRNA groups as target to treat the problem to all the patients.

It is known that many virus or bacterial infection will cause the immune system to attack certain cell/tissue/organ of the patient. Similarly, this method can also be used to stop the self immune attack therefore treat the corresponding disease. Another aspect of the current invention relates to methods to treat disease caused by virus infection, bacterial infection and parasites infection.

When the virus infect cell, the cell will present certain viral component (e.g. viral antigen) on the cell surface, which will later be recognized and eventually the cell will be killed by the killer T cell to stop the virus keep on replication in the host cell. However, this natural mechanism may not be enough. Therefore, the similar idea as described in the auto immune method described in the current inventions can be used; in brief, the affinity molecules (e.g. antibody, virus entry inhibitor, aptamers) that can bind with the viral component/antigen on the cell surface or specific marker of the infected cell presented on the cell surface will be coupled with toxin or its precursor or cell killing/inactivating/inhibiting agent as well as siRNA for virus or this target cell; and these conjugate can be applied to the host to selectively kill/inactivating the infected cell or virus. For example, antibody specific to gp120 coupled with ricin can be used to kill the HIV infected T cell. The affinity molecules can also be molecules bind to other cell surface marker of the infected cell or molecules can be readily uptaken by the cell. This method can also be used for treating other virus infection such as HBV, HCV and etc.; and as well as some bacterial/parasite infection such as malaria as long as the infected cell can present unique surface marker such as their protein. Other molecules such as cell membrane crossing agent can also be incorporated into the conjugate to maximize the therapeutical effect.

When the cell are infected with virus, it sometime produce unique surface maker. This unique marker can also be as the target for the affinity group of the current invention.

A blood purification/dialysis step can be performed before the above killing/inactivating/inhibiting treatment to remove the virus or bacteria or parasite or infected cell or their components (e.g. their antigen) in circulation. The protocol can be readily adopted from the methods treating immune disease described above. This step can reduce or eliminate the generation of immune complex formed, which may be toxic to the patients. In some embodiments, a blood purification/dialysis step can also be performed before the treatment as well to remove the self-antibodies generated by the patient against pathogens in circulation. It will reduce the competing of these self-antibodies with the later added therapeutics. The protocol can be readily adopted from the methods treating immune disease described above. Similarly, additional blood purification can be performed after the killing/inactivating/inhibiting treatment to remove the resulting binding complex (e.g. immune complex formed by the residual virus and the inactivating agent) in the blood.

In one example, patient having HIV infection is first treated with blood purification method to remove the circulating HIV particle and gp120 protein in the blood. The blood of a patient passes through a hollow fiber based plasma separator. The pore size of the membrane of the hollow fiber is 0.5 um, which is enough to allow the HIV particle to pass. The plasma part passes through a HIV/gp120 removal column (e.g. a column filled with 50 ml 90 um diameter CNBr-activated Sepharose™ 4B bead coupled with antibody against gp120 from goat, 10 mg antibody/ml capacity) and then the treated plasma is combined with the blood cells from the plasma separator and is sent back to the patient. The blood flow rate is 150 ml/min and the treatment continues for 2 h. Alternatively, whole blood without plasma separation is used to pass a HIV/gp120 removal column (e.g. a column filled with 100 ml 150 um diameter CNBr-activated Sepharose 4B bead coupled with antibody against gp120 from goat having 10 mg antibody/ml capacity; or 100 ml 300 um diameter CNBr-activated Sephadex G-50 coupled with antibody against gp120) to further remove the circulating HIV infected cells expressing gp120 besides the HIV virus and free gp120 in the blood. The blood flow rate is 150 ml/min and the treatment continues for 2 h. 2 hours after the blood purification, 3B3-PE (PLoS Pathog. 2010 June; 6(6): e1000803) is given to the patient at three intravenous doses in a week (20 ug/kg) to inactivate the HIV infected cell. Optionally after the injection of the drug, A C1q blood purification column can also be used to remove the formed circulating immune complex containing the 3B3-PE in the blood. Before the 3B3-PE is given, optionally the patient can also be treated with a blood purification to remove the circulating antibody against gp120 using a column filled with gp120 coated Sepharose 4B beads. This step will eliminate the antibody in the blood which may compete the binding of 3B3-PE with the target cells.

Examples of toxin/cell inhibitor/inactivator in the current inventions include but not limited to any agent that can kill the cell or inhibit the cell's normal or specific function (e.g. producing certain molecules such as protein (e.g. antibody), replication, differentiation, growth, develop into mature cell or other type of cell). They could be radio isotope, proteins, small molecules, siRNA, antisense molecules, enzymes and etc. Examples of them include NK cytotoxic factor, TNF such as TNF-α and TNF-β (LT), perforin, granzyme, cell apoptosis inducer/activator, free radical generating agent, cell membrane damaging agent, lipase, protease, hydrolase, toxic agent, chemotherapy agent, siRNA or antisense nucleic acid for the host cell's normal function, cytotoxic agent and etc. They can be made to be in precursor type or inactive type and only become active after they bind with target cell or been taken by the target cell, e.g. antibody-donomycin conjugate similar to the antigen-donomycin conjugate described above.

The toxin or its precursor or killing/inactivating/inhibiting agent can also be agent targeting the virus or bacteria or parasites so the conjugate can be used to selectively kill/inactivating the virus or bacteria or parasites instead of the host cell. For example, they could be anti viral drug for virus, antibiotics for bacterium, anti parasites agent for parasites, radio isotope, free radical generating agent, pathogen membrane damaging agent, pathogen toxic agent, lipase, protease, hydrolase, siRNA or antisense nucleic acid for the pathogens and etc. They can made to be in precursor type or inactive type and only become active after they bind with target pathogen or been taken by the target pathogen. For example, humanized antibody against E coli coupled with endolysin or polymyxin can be used to treat E coli infection. It can be injected to the blood for the treatment.

Furthermore, the toxin or its precursor or cell (or pathogen) killing/inactivating/inhibiting agent can also be a drug delivery system. The affinity group such as antibody or antigen is linked with the drug delivery system. The drug delivery system contains the means that function as toxin or its precursor or killing/inactivating/inhibiting agent. For example, the drug delivery system can be a polymer (e.g. poly lysine) coupled with multiple donomycin molecules, the antibody to gp120 is also coupled with this polymer. In another example, liposome contains ricin A chain molecules and the surface is coated with antibody against gp120. These examples can be used to treat HIV infection. Other drug delivery system such as micro particle, nanoparticle is also suitable for the current invention. This type of conjugates can be used to treat pathogen infection or auto immune disease.

When used to treat infection caused by virus, bacterial or parasite that are not inside the host cells, the affinity group need to target the unique surface marker of the virus, bacterial or parasite, e.g. their surface protein, antigen or membrane transporter. The affinity groups can be antibody or small molecules that can bind to their surface or substrate for their surface transporter or molecules that can be readily uptaken by the pathogens, e.g. antibody against their surface components (antigen), small molecules bind with surface protein (e.g. virus entry inhibitor), lectin specific to certain pathogen, certain antibiotic having affinity to pathogen surface.

In one example, a small molecule HIV virus entry inhibitor that can bind with gp120 is coupled with donomycin. Because it is a small molecule, it can be used orally to kill the HIV infected host cell. The small molecule HIV virus entry inhibitor that can bind with gp120 can also be coupled with a membrane disrupting agent so it will be able to kill the HIV virus directly.

The said killing/inactivating/inhibiting agent can also be the protein/proteins from the complement system or fragment of them or their mimics. For example, it could be a C1q or activated C1q or C3b or C3bBb or C3-convertase or C5-convertase or the membrane-attack complex; or their mimics or molecules having similar function or combination of them. When they are coupled with the affinity groups, the chemotaxis, phagocytosis or lysis of the pathogen bound with the affinity groups will be enhanced. If the affinity groups are not antibodies (e.g. aptamers), the Fc fragment of IgG can also be coupled with the affinity group or the said killing/inactivating/inhibiting agent to enhance the phagocytosis/lysis of the pathogen.

The said killing/inactivating/inhibiting agent can also be a molecule/molecules from pathogen-associated molecular patterns, or molecules selected from superantigens (SAgs).

When it is used to kill the infected cell, marker molecules of the apoptotic cell (e.g. a variety of intracellular molecules on the cell surface, such as Calreticulin, phosphatidylserine, Annexin A1 and oxidised LDL.) can also be used to couple with the affinity groups instead of the killing/inactivating/inhibiting agent. Therefore these infected cells will be taken up by macrophages.

Administration of the dimer or oligomer of IgG specific to certain pathogen will also provide better anti pathogen effect since the dimer or oligomer form of IgG will favor the complement system activation.

This method can also be used to treat HIV or other virus/bacterial infection that involve the production of harmful antibodies. The infected cells will present certain antigens of pathogen on their surface. For example, both gp120 and antibodies against it are necessary for HIV disease progression. Removing either gp120 or the antibodies against it will stop disease progression and allow for immune system reconstitution; first the patient with HIV can be treated with blood purification to remove the gp120 antibody as well as the HIV virus and gp120 protein in the blood, next, the patient will be treated with Immudel-gp 120 from Institute for Applied Biomedicine or the like to eliminate antigp120 antibodies producing cells, which is accomplished by the selective destruction of the B cells which produce them. The B cell clonal toxin, a hot antigen suicide agent compound, is used to selectively eliminate gp120-reactive B cells. The detailed procedure can be found in the related reference.

There are many drugs take effect by bind with the surface marker of pathogens or human cells. Examples of these kinds of drugs include but not limited to antibody-drug conjugates, affinity ligand-drug conjugates and virus entry inhibitors. Therefore similar to the method described above, a blood purification treatment can be performed to remove the circulating antigens/pathogens/cells having this surface maker and other substance in the blood that can bind with the drug with high affinity before these types of drug is given to the patient. This will minimize the side effect such as those caused by generating potential harmful immune complex, reduce the dosage for the drug and increase the drug efficacy. One method is to pass the blood or plasma through solid phase coated with drug or part of the drug or it's mimic in the extracorporeally circulating treatment. Other methods such as less selective plasmapheresis, apheresis or hemofiltration can also be used as long as the blood part containing these circulating antigens/pathogens/cells can be removed. Without removing these circulating antigens/pathogens/cells, the drug will bind with them to form a binding complex (e.g. an antibody-antigen immune complex if the drug contains an antibody part) which could be harmful. The drug can also bind with the circulating soluble antigen molecules (e.g. soluble gp120 in the blood of HIV patient) or other molecules in the blood having high affinity to the drug, to compete with the drug binding with its desired target (e.g. the pathogens/cells not in the blood) to reduce the drug efficacy. If they are removed, the drug will be more potent because the amount of target accessible drug is higher, and sometimes less drug can be used to reduce the side effect. Even if the desired target (pathogens/cells) is in the blood, removing significant amount them from blood before the patient is given the drug is also beneficial because the drug is more effective in treat the residual target and sometimes less drug can be used to reduce side effect. Preferably the drug is given to the patient before significant amount of circulating antigens/pathogens/cells is reproduced in the blood after the blood purification.

For example, antibody-drug conjugates (ADCs) are a type of targeted therapy, used for many diseases including cancer. They often consist of an antibody (or antibody fragment such as a single-chain variable fragment linked to a payload drug (often cytotoxic). One can use blood purification to remove the antigen in the blood before the antibody-drug conjugates. Furthermore, the blood purification can also be performed after ADCs is given to remove the resulting immune complex in the blood. In one example, Brentuximab vedotin is an antibody-drug conjugate approved to treat anaplastic large cell lymphoma (ALCL) and Hodgkin lymphoma. The compound consists of the chimeric monoclonal antibody Brentuximab (which targets the cell-membrane protein CD30) linked to antimitotic agent monomethyl auristatin E. The patient is first treated with blood purification to remove the CD30 and cells expressing CD 30 in the blood (e.g. the blood of a patient passes through a CD 30 removal column such as a column filled with 100 ml 150 um diameter CNBr-activated Sepharose™ 4B bead coupled with Brentuximab or 100 ml 300 um diameter sephadex beads coupled with Brentuximab, at a flow rate of 150 ml/min for 2 h). Alternatively, the patient can be treated with blood cell separator (apheresis) to remove most of the white blood cells in which the cells expressing CD 30 is inside. Next Brentuximab vedotin is given to the patient for the treatment. In another example, Enfuvirtide is an HIV fusion inhibitor, which binds to gp41 preventing the creation of an entry pore for the capsid of the virus, keeping it out of the cell. A patient with HIV infection is first treated with blood purification to remove the HIV and free gp41 in the blood. The blood of a patient passes through a hollow fiber based plasma separator. The pore size of the membrane of the hollow fiber is 0.5 um, which allow the HIV particle to pass. The plasma part passes through a column filled with ml 100 um diameter Sepharose™ 4B beads coupled with antibody against gp120 and antibody against gp41) and then the treated plasma is combined with the blood cells from the plasma separator to form the cleaned blood. The cleaned blood is sent back to the patient. The blood flow rate is 150 ml/min and the treatment continues for 2 h. Next the patient is given the Enfuvirtide as treatment either using the standard protocol or reduced dose.

Another aspect of the current invention relates to a method for reducing the viral load by removal of viruses or its fragments or its components or virus infected cell thereof from the blood by extracorporeally circulating blood through solid phase immobilized with affinity molecules having affinity for viral components. Passage of the fluid through the solid phase causes the viral particles and/or virus infected cell to bind to the affinity molecules thereby reducing the viral load in the effluent. Similarly, other pathogens such as bacteria and parasite (e.g. malaria when the red blood cell is broken) can also be removed using with solid phase having affinity molecules with affinity for their components if these pathogens are in the blood.

The solid phase support for blood purification could be a column, a membrane, a fiber, a particle, or any other appropriate surface, which contains appropriate surface properties (including the surface of inside the porous structure) either for direct coupling of the affinity molecules or for coupling after modification or for surface derivatization/modification. If the solid support is porous, its inside can also be used to present the binding affinity molecules.

When the virus infect cell, the cell will present certain viral component (e.g. viral antigen) on the cell surface. So the solid phase support coupled with affinity ligand for virus (preferably the viral antigen on the infected cell surface) will also bind with the cell infected with virus besides the virus. Therefore therapeutical effect to treat viral infection can also be achieved by removing the virus harboring cells from the blood.

In some embodiments, the blood passes through hollow fibers within a cartridge, wherein affinity molecules for virus are immobilized within a porous wall portion of the hollow fiber membrane. Examples of the virus include HIV-1, HBV and HCV. Examples of affinity molecules are antibodies, aptamer, lectin or virus entry inhibitors for these viruses. The affinity molecules can also be attached to a solid matrix and be placed within the blood purification cartridge but outside the porous exterior portion of the hollow fiber. A means that can help the liquid outside the hollow fiber moving (such as pump or stirring device) can be applied to the liquid to increase the diffusing rate. One example of the solid matrix is sepharose. Examples of the hollow fiber membrane can be found in U.S. Pat. No. 6,528,057 and U.S. Pat. No. 7,226,429. The blood purification devices and protocols can also be readily adopted from these patents and other blood purification references. The affinity molecules can also be attached to a solid phase matrix and be placed within the blood purification cartridge and the blood passes through the matrix directly without using hollow fiber. Means that can inactivate the virus such as UV, radiation, heat, microwave, light can also be applied to cartridge or the solid phase within to inactivate the virus inside.

In one example of the method of the present invention, blood is withdrawn from a patient and contacted with the ultra filtration membrane having affinity molecules. In some preferred embodiments, the blood is separated into its plasma and cellular components. The plasma is then contacted with the affinity molecules specific for the virus (or other pathogen) or their surface protein, to remove the virus or components thereof. Following removal of virion (or other pathogen) and/or free nucleic acid, the plasma can then be recombined with the cellular components and returned to the patient. Alternatively, the cellular components may be returned to the patient separately.

Means that can kill the virus or other pathogen can also be applied to the solid phase or the plasma part only. For example, low temperature (e.g. −10 degree) or high temperature (e.g. 40-60 degree) can be applied to the solid phase support (e.g. the column, filters, fibers and membrane) or the filter or the separated plasma part. Light (UV or visible light), microwave or radiation can also be applied. Preferably, the means to inactivate pathogen has some selectivity to pathogens over the normal plasma component. For example, if UV is used as means to inactivate pathogens, in some applications the preferred wavelength is the wavelength at which the nucleic acid has high absorption but protein has lower absorption, e.g. 260 nm. Because the virus will stay longer/trap in the solid phase/filter, they will be cool/heat/light or radiation treated much longer time, by carefully control the intensity of the treatment, the virus will be killed but the healthy cells/plasma component will still be alive/active because they pass through the solid phase/filter quickly. The flow speed, treatment intensity (e.g. temperature, light or radiation intensity) can be adjusted so that only the cells/pathogens stay on the solid phase for a long time will be killed. So even if the virus or other pathogens are released from the solid phase to the blood they still cannot cause new infection. One method to keep the virus stay longer in the inactivating device is to fill the cartridge of the inactivating device with solid phase support particle having many pore/cavity. The size of the pore/cavity is bigger than the size of the virus but smaller than the blood cell. So when the whole blood pass through the virus will be trapped inside the solid phase and take long time to get out but blood cells will flow away quickly. This mechanism is similar to that of the size exclusion chromatography. Therefore the virus can be treated longer to be inactivated. If photon such as IR, visible light or UV is used to kill the virus, photoactive agents (e.g. those used in photochemical pathogen inactivation for treating blood products) such as phenothiazine dyes, methylene blue, vitamin B2, psoralen (e.g. 8-MOP, AMT), agents used in photodynamic therapy such as photosensitizer can also be added to the blood to increase the virus/pathogen/infected cell inactivating efficacy. These agents can also be coupled with affinity ligand for the pathogen to increase their selectivity. They can be added to the whole blood or the plasma part. They can also be added to the patient or added to the blood/plasma after the blood is taken out. Furthermore, these agents can be removed from the blood/blood component after the pathogen inactivating treatment but before the blood/blood component is returned to the patient to reduce the potential side effect of these agents to the patient. For example, by passing the blood/blood component through a blood purification device filled with adsorbent (e.g. charcoal, absorption resin) that can absorb these agents or a blood dialyzer. There are many these types of devices and techniques available for blood purification/blood perfusion/blood dialysis to remove drugs in the blood. One can readily adopt them for the current application. For example, crosslinked agar entrapping attapulgite clay, Pall MB1 filter, Maco Pharma Blueflex filter or LeucoVir MB filter can be used to remove methylene blue in the blood or blood component. If only the plasma part is treated with virus/pathogen killing means (e.g. using a plasma separator to separate the blood cells and the virus containing plasma and then only apply the inactivating means to the plasma part), it may not be always required to remove the virus/pathogen from the plasma using solid phase adsorbent or filter although combining virus killing with solid phase adsorbent or double filtration will increase the therapeutic efficacy. There are many ways to separate plasma from whole blood such as using hollow fiber type plasma separator and many blood component separation devices based on centrifugation. Because many pathogens are in the plasma so treating the plasma only can also reach the pathogen reducing/inactivating effect and reduce the damage to the blood cell. If hollow fiber type plasma separator is used, the pore on the hollow fiber should be big enough to allow pathogen to pass through but not allow most blood cells to pass. In some embodiments, the plasma passes through a filtration device (e.g. a filter) to remove the pathogen inside (e.g. using Double-filtration plasmapheresis) and is also treated with said pathogen inactivating means after or before the filtration. The combination of filtration and pathogen inactivating will result in better therapeutical effect.

The treatment can be repeated periodically until a desired response has been achieved. For example, the treatment can be carried out for 2 hours every 3 days or every week. Thus in some examples, the essential steps of the present invention are (a) contacting the body fluid with the affinity molecule immobilized to an solid phase support (e.g. particles) under conditions that allow the formation of bound complexes of the affinity molecules and their respective target molecules; (b) collecting unbound materials; and (c) reinfusing the unbound materials into the patient.

These methods described in the current invention can also be used to treat other pathogen infection such as bacteria or parasite, as long as they are in the blood. The treatment can be done either in a continuous flow fashion or intermittent flow fashion. For example, the blood is withdrawn continuously and been treated continuously and returned to the patient continuously. In another example, certain volume of blood/blood component is withdrawn and been treated for certain period of time then return to the patient and then the next batch of blood/blood component is withdrawn for treatment. This will allow enough time for the pathogen inactivating. It can also be the combination of continuous flow/intermittent flow. For example, the blood passing through the plasma separator and adsorbent is done continuously but the pathogen inactivating and plasma returning to the patient is done in batch. If the whole blood withdrawing and return is done in an intermittent flow fashion, single needle/catheter in the body can be used for both withdrawing and returning blood in a time slicing fashion by doing them in different time interval.

In some embodiments, the blood or blood component passing through adsorbent is repeated a few times. For example, after the blood or blood component passing through a cartridge filled with adsorbent it is re introduced to the cartridge to allow it pass the adsorbent again before going back to the patient.

There are numerous methods for coupling a chemical to solid support. These methods are readily available from scientific journals, vendors that provide coupling reagents, or relevant websites. For example, chemicals containing a primary amine can be coupled to a solid support that is functionalized with a carboxyl group through the formation of amide bond; the formation of amide bond between the amine and carboxyl group is normally catalyzed with EDC [1-(3-dimethylaminopropyl)-3-ethylcarbodiimide hydrochloride] or other carbodiimide. The virus binding chemicals may need to be appropriately modified or derivatized to introduce a functional group that can be used for coupling while at the same time the modification or derivatization does not inactivate the virus binding activity. It is understood that the binding chemicals can be chemically or naturally coupled to another moiety that can be subsequently coupled to a solid support. In other examples, the virus binding chemicals themselves could used to form the solid phase support.

One aspect of the current inventions utilizes solid support having strong negative charged groups or coated with strong negative charged groups (e.g. sulfonic acid, sulphanic acid and sulfonate groups or their salts) to remove the virus. The poly anions used as virus binding chemicals include, but are not limited to, a copolymer of maleic acid and styrenesulfonic acid, a polymer of polyvinyl phthalate sulfate, sulfated polysaccharides (e.g. curdlan sulfate, dextrin sulfate, fucoidan, and pentosan polysulfate, dextran sulfate, heparin, heparin sulfate, carrageenan), polyvinylsulfate (PVS), and polyanethole sulfonate, their copolymers with acrylic acids and salts thereof. Most preferably these polymers have high density of sulfonic or sulfate functional groups or phosphate groups or carboxylic acid groups (e.g. poly acrylic acid, poly maleic acid). One example of polymers encompasses copolymers of maleic acid and styrenesulfonic acid. Another example of polymers encompasses polymers of polyvinyl phthalate sulfate, which can be mixed esters comprising phthalate and sulfate functional groups on a polyvinyl backbone, and which can be produced as an esterification product of polyvinyl alcohol by phthalic anhydride and sulfuric chloride. Each of these classes of compounds has a high density of acid functional groups. For copolymers of maleic acid and styrenesulfonic acid that are useful in the present invention, the molecular weight ratio of the maleic acid to the styrenesulfonic acid can be varied freely in almost any amount (e.g., molecular weight ratios are effective at from 9:1 to 1:9; 7:3 to 3:7; and at about 1:1). In one example, the molecular weight ratio of maleic acid to styrenesulfonic acid is about 1:3. The copolymers of maleic acid and styrenesulfonic acid (PSMA) can be made by well-known methods employing copolymerization of maleic acid with sulfonated styrene (e.g., Kobashi et al. U.S. Pat. No. 4,009,138), or by hydrolysis of a copolymer of maleic anhydrate and styrenesulfonic acid. The synthesis of copolymers of maleic anhydrate and styrenesulfonic acid is described by Bauman et al. (U.S. Pat. No. 2,835,655). They are also commercially available from Sigma-Aldrich, Inc. Other virus binding chemicals include lectin, antibody and aptamer.

These virus binding chemicals are immobilized on solid support to remove virus from blood. In example 1, coupling of PSMA to the particle can be performed as follows: 20 mg of amine coated silica or agarose particles (200 um in diameter) are washed three times with 0.1 M MES, pH 5.0 and again three times with deionized water. The particle wet cake is suspended in 0.5 mL of PSMA (Sigma-Aldrich) at 20 mg/mL in deionized water, followed by an addition of 0.5 mL of 20 mg/mL carbodiimide [1-655 Ethyl-3-(3-dimethyl-aminopropyl)-carbodiimide hydrochloride, EDC] in deionized water, which is prepared immediately before use. The pH is then adjusted to 7.5 with 0.1 M NaHCO₃ solution. The particles are rotated at room temperature for 2 hours.

Another 10 mg of EDC and 10 mg of NHS(N-hydroxysuccinimide) are added to the mix, followed by an overnight rotation at room temperature. The particles are washed 3 times with 10 mM HEPES buffer, pH 7.5, 5 times with deionized water and then suspended in 1.0 mL of deionized water. The reagent is now ready to be packed in a column for use for virus binding.

The solid support can also be derivatized/modified to have strong negative charged groups on its surface and inside (if it is porous). For example, the polystyrene beads can be sulfonated and the resulting beads will contain high density of styrenesulfonic acid for virus binding. In one example, Amberlite® IR120 resin in sodium form is used.

As described before, the solid support can contact with the whole blood directly or contact the plasma after the blood is being processed by a plasma separator (e.g. a hollow fiber separator) to remove the virus or its components.

Because sometimes the virus are coated with antibody in the blood, one can also use solid phase immobilized with antibody affinity molecules (e.g. protein A or virus antigen since each antibody has two binding sites) to capture the virus-antibody complex. Preferably, the affinity molecule has high affinity to the antigen-antibody complex, such as the complement molecule (e.g. C1q). In one example, the combination of C1q immune absorption column and the virus removal column in the current invention is used in blood purification to treat virus infection as it can remove both the free virus and the antibodies bound virus particle in the blood. Alternatively, the absorption column contains both adsorbent coated with C1q and adsorbent coated with affinity ligand (e.g. antibody) for the virus surface molecules and adsorbent coated with virus surface antigen; or the adsorbent that are coated with both C1q and said affinity ligand. Other column such as TR350 or PH350 can also be used to remove antigen-antibody complex although they are less specific. Many molecules that can bind with antigen-antibody complex are described in U.S. patent application Ser. No. 10/803,246, such as C1q derived molecule, gC1q, gaC1q, gbC1q or gcC1 q, polypeptide is structurally or functionally similar to the C1q A, B or C chains, molecule has higher binding affinity to a Glu-X-Lys-X-Lys motif than antibody globular head wherein X is an amino acid, C1q fragment/analogues/direvtives and etc. They can be used as affinity molecule for the current invention. In one example, the microparticle solid phase support is coated with both c1 q and affinity ligand for virus.

Because sometimes the virus (e.g. HBV, HCV) bind with lipoproteins, device and methods used for lipoprotein apheresis can also be used in combination with the virus removal/inactivating device/methods described above to further remove the virus. For example, additional heparin induced extracorporeal lipoprotein precipitation can be used to further remove the virus-lipoprotein complex. Lipoprotein removal cartridge such as dextran sulphate cellulose columns and LIPOSORBER System can connected in the extracorporeally circulating blood path to remove the virus. The solid phase adsorbent used in lipid filtration/lipoprotein removal and also be filled in the virus removal cartridge contains other solid phase adsorbent coated with affinity ligand (e.g. antibody) for virus to form a mixed adsorbent cartridge to be used for virus removal. The solid support having strong negative charged groups previously described can also be used to remove virus-lipoprotein complex besides pure viral particle.

The solid phase support coated with different affinity molecules for virus/pathogen or their immune complex or their lipoprotein complex can also be combined in the blood purification to remove virus/pathogen. They can be a mixture of several different solid phase supports each having their unique affinity molecules or simply immobilizing several types of affinity molecules on the same solid phase support. One can also use several different type of pathogen removing cartridge in serial to reach maximal pathogen removing effect.

In some embodiments, the blood is withdrawn from the patient and extracorporeal circulating is established. The blood is separated into plasma component containing the pathogen and cellular component by passing through a cartridge. In one example, the cartridge contains many hollow fiber made of polysulfone membrane. The total area of the membrane is 0.5 m² and the pore size of the membrane is 0.2˜0.6 um. One ends of the cartridge has blood inlet and another end has blood outlet to connect with the blood from artery and return blood to the vein and pass the blood through the hollow fiber. Optionally, inside the cartridge but outside hollow fiber is filled with adsorbent particles (size>the pore size of the membrane) having affinity to the target pathogen surrounding the hollow fiber. The design of the cartridge can be readily adopted from prior art such as those described in the U.S. patent application Ser. Nos. 12/282,152, 11/756,543 and their cited references.

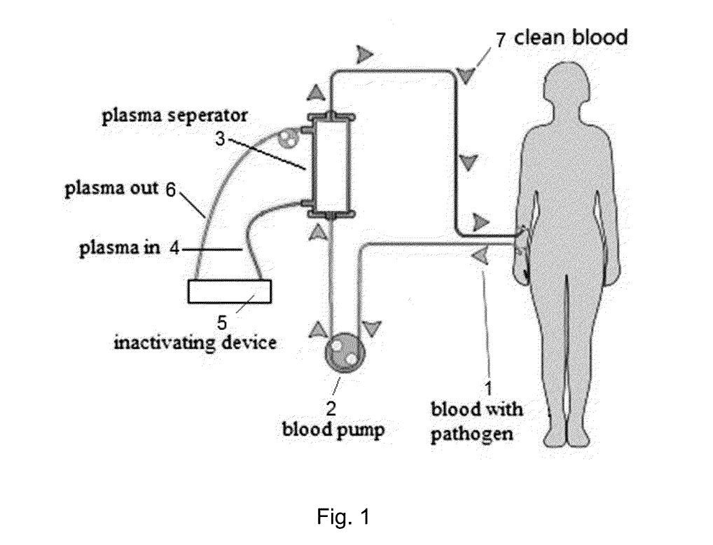

In example 2 shown in FIG. 1, the blood 1 of a patient with HIV infection passes through a plasma separator 3 via a blood pump 2. The plasma part 4 passes through an UV irradiation virus inactivating device 5 and then the treated plasma 6 is combined with the blood cells part in plasma separator 3. The clean blood 7 is sent back to the patient. Alternatively the clean plasma 6 can be sent back to the patient directly without combining with blood cells or be combined with the blood cells outside the plasma separator and then sent back to the patient. Additional HIV virus absorption device or virus filtration device can also be added. For example, a HIV absorption device (e.g. a cartridge filled with 20 um diameter solid phase adsorbent particle coated with affinity ligand for HIV virus) or a virus filtration device (e.g. a filter having pore size of 60 nm since HIV virus has a size of 100 nm in diameter) can be connected to the plasma in or plasma out path (or the path back to the patient for HIV absorption device).

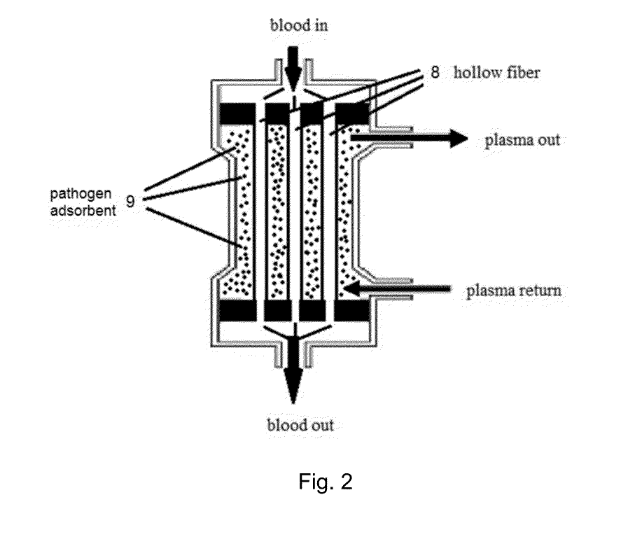

The FIG. 2 shows a plasma separator filled with pathogen adsorbent for virus removal. In an example to treat a patient with HCV (or HBV) infection, first the blood of the patient with HCV (or HBV) infection is withdrawn from the patient and extracorporeal circulating is established. The blood is separated into plasma component containing the virus and cellular component by passing through a cartridge shown in FIG. 2. The cartridge contains many hollow fiber 8 made of polysulfone membrane. The total area of the membrane is 1 m² and the pore size of the membrane is 0.5 um. One ends of the cartridge has blood inlet and another end has blood outlet to connect with the blood from artery and return blood to the vein. The blood passes through the hollow fibers. Inside the cartridge is filled with pathogen adsorbent particles 9 (size>the pore size of the membrane) having affinity to the target pathogen surrounding the hollow fiber. One example of the adsorbent particle is 90 um diameter Sepharose 4B coupled with PSMA or 90 um diameter Sepharose 4B coupled with antibody against HCV (or HBV) surface antigen. The plasma from plasma out path can further passes through a virus inactivating device (e.g. UV irradiation or isotope irradiation) and then returns to the cartridge. Additional photoactive agent (e.g. those used in photochemical pathogen inactivation) or photosensitizer can be added to the plasma before the plasma goes into inactivating device and be removed with charcoal after it go out from the inactivating device. The plasma going in and leaving the inactivating device can be done in a batch format (e.g. by adding valves in the path and open/close the valve after certain period of time) to ensure enough stay time of the plasma in the inactivating device for desired treating time.

In example 3, the extracorporeal blood circulating is established for a patient with HCV infection. The blood passes through a plasma separator at the flow rate of 200 ml/min. The separated plasma goes into and passes through a flat UV transparent container (e.g. an inner size 10×10×1 cm quartz box). The box is irradiated with UV light of 253 nm at the intensity of 60 uW/cm². The plasma travel from one end of the box (plasma inlet) to another end of the box (plasma outlet) in 30 seconds continuously. The treated plasma then is combined with blood cells from the plasma separator and goes back to the patient. The entire treatment takes 2 hours. If desire, the treatment can be repeated several times, e.g. once every 3 days. After the plasma is treated with UV radiation at the above intensity and wavelength, more than 95% HCV virus in the plasma can be inactivated based on the result from virus culture test. Other radiation intensity, wavelength and flow rate and time can also be applied, e.g. 220-280 nm UV, 30 uW˜3000 uW/cm², 20 seconds to 120 seconds radiation time (the plasma stay time in the radiation path, which is determined by flow rate, shape and size of the radiation path, e.g. the said quartz box). The parameter selected need to provide high pathogen inactivation rate yet low normal plasma protein inactivation rate. For different pathogen, these parameters can be determined experimentally. During the treatment, photoactive agents (e.g. those used in photochemical pathogen inactivation for treating blood products) such as phenothiazine dyes, methylene blue, vitamin B2, S59, psoralen (e.g. 8-MOP, AMT), agents used in photodynamic therapy such as photosensitizer can also be added to the blood or plasma to increase the virus/pathogen/infected cell inactivating efficacy. They can be added either to the plasma directly before the radiation or into the whole blood outside the patient or given to the patient orally or by injection. They can also be coupled with affinity ligand for the pathogens to increase their specificity. The amount added need to be sufficient to inactivate the pathogens under the applied radiation. For example, vitamin B2 can be added to the plasma to reach the concentration of 100 uM and the radiation intensity is 1 mW/cm² at the wavelength of 260 nm-370 nm or 450 nm. A vitamin B2 absorbing cartridge (e.g. a column filled with 100 g of agarose (or gelatin) coated activated charcoal particle) is placed in the downstream of the radiation path to prevent excess vitamin B2 going to the patient. Besides a box shape container, other type of radiation path can also be used such as a spiral tube surrounding a UV lamp. The plasma can either join the blood cell outlet of the plasma separator before going back to the patient or return to the patient directly without combing with the blood cells in which case the plasma separator may not need to have a plasma inlet. Alternatively, heating can be used to inactivating virus instead of UV radiation. For example, the box is placed in a microwave generator and the plasma inside is heated to a temperature of 56 degree. After the plasma is heated at 56 degree, more than 95% HCV virus in the plasma can be inactivated based on the result from virus culture test. Other temperatures can also be used such as those between 50-70 degree. Furthermore, cartridge filled with HCV adsorbent or a filter with 60 nm pore size can be placed in the downstream of the radiation path to further clean the plasma. Examples of HCV adsorbent include solid phase support coupled with affinity ligand for HCV/their immune complex (e.g. 50 ml 90 um diameter Sepharose 4B beads coupled with a 1:1 molar ratio mixture of C1q and antibody (or lectin) against HCV surface protein).

In example 4, the extracorporeal blood circulating is established for a patient with HIV infection as shown in FIG. 1. The blood passes through a plasma separator shown in FIG. 2 at the flow rate of 100 ml/min. The separated plasma goes into and passes through a flat UV transparent container 5 (e.g. an inner size 10×10×1 cm quartz box). The box is irradiated with UV light of 260 nm at the intensity of 200 uW/cm². The plasma travel from one end of the box (plasma inlet) to another end of the box (plasma outlet) continuously. The treated plasma is then combined with blood cells and goes back to the patient. The entire treatment takes 3 hours. If desire, the treatment can be repeated several times, e.g. once every week. After the plasma is treated with UV radiation at the above intensity and wavelength, more than 95% HIV virus in the plasma can be inactivated based on the result from virus culture test. The plasma separator is filled with HIV adsorbent. The HIV adsorbent contains a mixture of 30 ml of 90 um diameter Sepharose 4B particle coupled with antibody against HIV gp120 and 30 ml of 90 um diameter Sepharose 4B particle coupled with C1q.

The current invention also discloses methods and devices for ablation of circulating cells/pathogens, which are the modification on those described in U.S. patent application Ser. No. 12/227,843. U.S. patent application Ser. No. 12/227,843 describes methods and devices for the extracorporeal ablation of target cells circulating in blood of an organism. Exogenous material introduced into the blood preferentially associates with target cells (e.g. cancer cells, bacteria, viruses) in the blood. An extracorporeal continuous flow pathway accesses the patient's blood to apply an external energy source to the blood at an ex vivo ablation device in a portion of the extracorporeal continuous flow pathway. The exogenous material interacts with the applied energy so as to result in the damage or death of the target cells. The blood is then returned to the body in a continuous-flow pattern. The current invention describe modifications on these methods/devices in said prior art patent. These modifications can be applied independently or be applied in any combinations.

The first modification is to use separated blood components to receive energy instead of whole blood. In the said prior art application (application Ser. No. 12/227,843) whole blood is used to receive energy. This will cause all the blood components absorb the energy therefore may be damaged. In the current invention, the whole blood is first separated into different blood components and only the selected component is being treated with energy. The selected component needs to contain the target cell/pathogen. Further more in some applications said exogenous material can be added only to the selected component. If the target cells for ablation (inactivation) is in plasma (e.g. some virus, bacteria and parasites), the plasma can be separated from the whole blood and be treated with energy. In some embodiments the exogenous material can be added only to the plasma before the energy treatment. The blood can first pass through a plasma separator as previously described and then the plasma part is mixed with suitable amount of exogenous material and then only this plasma part receive the energy. The blood cell components can be returned to the body directly or be combined with the plasma that has been treated with energy and then go back to the body. The procedure and device can be readily adopted from the prior art patent and those described in the current application. If the target cells for ablation (inactivation) is in human cell (e.g. circulating cancer cells), the withdrawn whole blood can be first separated into to several parts by using blood cell separation means such as apheresis. Only the part contains a great number of target cell will be treated with energy and optionally only this part is added with said exogenous material. For example, in order to separate the CTC (circulating tumor cells) from the whole blood, many methods can be applied such as leukapheresis, size based filtration, centrifuge and elutriation. Many blood cell separation devices can be used such as verities of blood cell separator, e.g. cs3000plus blood cell separator, COBEVR Spectra system and the Elutra system (Caridian BCT). After being processed with blood cell separator, most CTC will stay within the leukocyte component. In some cases CTC will be in the mononuclear cells component. In some cases the CTC will stay in the monocyte portion. One can readily isolate these components with suitable device. Next the portion containing the CTC (e.g. the monocyte portion or the mononuclear cell portion or the entire leukocyte portion) will be treated with energy. In some embodiments only this portion is added with exogenous material before being treated with energy. Other blood components can be sent back to the body directly after the separation or be combined with the energy treated blood component then return to the body. Optionally these other blood components can also pass through a blood purifier or be treated with a different CTC/pathogen inactivating means.

The second modification is that additional means is applied to remove the exogenous material from the blood or blood component after energy being applied and before the blood/blood component return to the body. In the prior art application, the exogenous material is also returned to the body which may cause side effect to the body. In the current application, these agents can be removed from the blood/blood component after the energy treatment but before the blood/blood component is returned to the patient to reduce the potential side effect of these agents to the patient. For example, by passing the blood/blood component through a blood purification device filled with adsorbent (e.g. charcoal, absorption resin, solid phase support coupled with affinity ligand specific to these agents) that can absorb these agents or a blood dialyzer. There are many these types of devices and techniques available for blood purification/blood perfusion (hemoperfusion)/blood dialysis to remove drugs in the blood. One can readily adopt them for the current application. A blood dialyzer using half permeable membrane or filter membrane can selectively remove the exogenous material from the blood or blood component because of the difference in their molecular weight or size. The adsorbent filled blood purification device can also be used to remove these exogenous material when the blood or blood component pass through the device. The absorption can either be non selective or selective. For example, charcoal and absorption resin are less selective adsorbent. Solid phase coated with affinity molecule specific to the exogenous material is can be used as adsorbent to selectively remove the exogenous material. In the prior art application, ligand is attached to the exogenous material, so the affinity molecule can either target the said ligand or said exogenous material. For example, in the prior art antibody coupled photosensitizer is used so either protein A or antibody against photosensitizer can be coated on the solid phase support to selectively remove the antibody coupled photosensitizer from the blood or blood component. After the whole treatment is complete, additional dialysis or blood purification can also be conducted to remove the added exogenous material/ligand from the blood. A variation is that the whole treatment is performed as described in the prior art (the blood returned to the patient directly without removing the added reagents), but after the completion of the treatment, additional blood dialysis or blood purification is conducted to remove the added reagents (e.g. exogenous material and ligand) from the blood.

The third modification is that instead of continuous flow, intermittent flow can be applied to the whole process or part of the process. In the prior art application energy is applied to the whole blood continues flow path way therefore the blood may not get enough time to be treated with energy for maximal effect. In the current invention the blood or blood components being treated with energy can be in an intermittent flow (batch) fashion to enable they get desired time length of energy treatment (e.g. 5-10 min for photo dynamic treatment). For example, the flow in the energy receiving area is stopped when certain amount of blood/blood components is being energy treated. After certain period of time the treatment is finished and the blood/blood component is released from this area and the next batch come into the energy treatment path. The adding of exogenous material to blood or blood component and incubating of it with blood or blood component can also be done in an intermittent flow fashion so the exogenous material will have enough time to associate with the target cell (e.g. 2-5 min). Other process such as blood withdrawn, blood returning and optionally blood separation can be either in a continuous flow fashion or intermittent flow fashion. For example, the blood is withdrawn continuously and separated into plasma continuously and returned to the patient continuously. In another example, certain volume of blood/blood component is withdrawn and been treated for certain period of time then return to the patient and then the next batch of blood/blood component is withdrawn for treatment. In another example, the blood passing through the plasma separator and adsorbent is done continuously but the pathogen inactivating and plasma returning to the patient is done in batch. A buffer zone can be provided in the flow path to accommodate the changing volume.

In some embodiments, a pathogen removal device or cell (e.g. CTC) removal device can be placed before or after the energy treating device in the extracorporeally circulating path. The pathogen removal device or cell removal device is described throughout the current applications.

In the prior art application, the exogenous material can be coupled with affinity ligand to the target cell/pathogen. However, the exogenous material coupled with affinity ligand to the target cell/pathogen can still be used as exogenous material and essentially a new exogenous material. For example, photosensitizer such as Photofrin or Levulan can be coupled with antibody against CTC or HIV and then be used as exogenous material for corresponding application. Photofrin or Levulan or nano particle TiO2 coupled with folic acid or virus entry inhibitor can also be used as exogenous material. When the virus infect cell, the cell will present certain viral component (e.g. viral antigen) on the cell surface. So the exogenous material coupled with affinity ligand for virus (preferably the viral antigen on the infected cell surface) will also kill the cell infected with virus besides the virus by selecting the exogenous material that can damage both human cells and virus. Therefore therapeutical effect to treat viral infection can be achieved by kill the virus harboring cells.

Before removing the pathogens/infected cell from the blood and/or inactivating the pathogens/infected cells with extracorporeally circulating blood as described above, one can withdraw a small amount of blood (e.g. 10˜50 ml) from the patient and test it with the method to be used in a small scale for its in vitro efficacy of removing/inactivating the pathogens/infected cell. Only if significant amount of pathogens/infected cell in the blood sample is removed or inactivated the full scale treatment using this method with extracorporeally circulating blood will be used to the patient. Otherwise a different method will be tested with a small amount of blood to find out the best method to remove/inactivate the pathogens/infected cell for the patient. Alternatively a small amount of blood is withdrawn and divided to several portion, each is treated with a different pathogen removal/inactivating method in vitro and the results are compared, the method shows the best efficacy will be used for the treatment to the patient if they have similar safety profile. If they have different safety profile, the method having high efficacy yet low side effect will be used. Because only a small amount of blood (e.g. 1˜200 ml) is tested instead of liters of blood during extracorporeally circulating blood, a smaller scale of device/reagent and a shorter time can be used instead. Part or the whole procedure of the method to be used to the patient will be performed to the blood sample to predict its efficacy during extracorporeally circulating blood. If no significant amount of pathogens/infected cell (e.g. <15%) is reduced/inactivated using this method when testing this small amount blood sample, this method will not be used. The method will be used to the patient only when significant amount of pathogens/infected cell in the blood sample is removed or inactivated (e.g. in some cases, >25% is required; in another cases, >50% is required) for the small amount of blood sample. For example, 20 ml blood can be withdrawn from the patient and a smaller size cartridge containing a small amount of pathogen adsorbent can be used in vitro for this blood sample to predict if a regular size cartridge with more pathogen adsorbent should be used for whole volume blood extracorporeally circulating treatment. The size of the cartridge and amount of pathogen adsorbent for the test can be reduced accordingly based on the difference between the volume of the blood sample and the blood volume of the patient. For example, one can use a small column filled with 1˜2 g of pathogen adsorbent for 20 ml blood in vitro test if the cartridge for the patient treatment containing 100 g pathogen adsorbent. In one example, during the in vitro test, 30 ml blood is withdrawn from the patient. 15 ml blood sample passes though the small column filled with 1 g pathogen adsorbent and another 15 ml blood does not. Then the amount of pathogens in the two samples are checked. If more than 50% of the pathogen in the blood sample treated with the small column is removed, the corresponding treatment cartridge can be used for the patient. It is understood the structure of the device, the parameter and the procedure for the in vitro test need not to be exactly identical to that used to treat the patient, e.g. the size, time, flow rate can be adjusted to fit the in vitro test format as long as the in vitro test can give a prediction of the efficacy of the treatment for the patient. Similarly, the pathogen inactivating method such as those using drug or exogenous material or physical means as previously described can also be tested in vitro with a small amount of blood sample from the patient before certain method is used for this patient. The combination of several methods/devices (size reduced if necessary) can also be tested in vitro using small amount of blood sample from the patient and if the overall pathogen/infected cell removing/inactivating efficacy is satisfactory, the combination will be used to treat the patient.

Virus infection can cause the defense system response to resist the viruses and protect host from further viral infection, such as increase body temperature, secrete cytokines and produce the antibodies. Fever can prevent virus replication, the cytokines can induce natural killer cells and macrophage to kill the virus and antibodies can neutralize viral infection. According to the different responses, one can distinguish viral pathogens. Detection of viral antigens is a big challenge since there are multiple tests needed especially for blood products. The universal and nonspecific biomarker(s) is needed to ensure safety of the blood product manufacture/usage and reduce test cost and time. Here, a new method is invented to evaluate safety of blood products by using ELISA or RT real time to detect products from nonspecific immuno response. Interferon is such a marker for indication of the status of viral infection in the blood products. There are 3 kinds of interferons (IFNs), IFNa, IFNb and IFNg. Among them, IFNa, and IFNg are two important markers in blood products and both produced from lymphocytes (especially memory CD4+ T cells. In normal condition, these cells do not produce these two kinds of cytokines and no such molecules can be detected in the blood. After virus infection, especially RNA viruses, IFNa increases quickly in early infection. The IFNa increase is dependent on viral double strain RNA and DNA; IFNg increase is caused by viral antigens and it is a later marker for the viral infection. Besides virus infection, some bacterial infections also can increase these two interferon secretions. Not only IFNs increase but also other cytokines increased too, such as interleukin-5,8,15 and 18, as well as, inducible protein 10, which can be used as a markers to evaluate blood product. If some of those factors increase in blood production, the blood products have possible virus, bacterial contamination or other abnormal condition exist, and the blood products may be not suitable to use. The IFN-α proteins are produced by leukocytes. They are mainly involved in innate immune response against viral infection. They come in 14 subtypes that are called IFNA1, IFNA2, IFNA4, IFNA5, IFNA6, IFNA7, IFNA8, IFNA10, IFNA13, IFNA14, IFNA16, IFNA17, IFNA21. Interferon-gamma (IFN-γ) is a dimerized soluble cytokine that is the only member of the type II class of interferons. For ELISA test, the serum can be tested used ELISA test kits (most ELISA Kits are commercial available) for these cytokines. For RT real time PCR test, the blood cells can be used to obtain mRNA that can be used to produce cDNA. Related primers can be used to amplify those cytokine gene expressions. Usually, these increased mRNA levels indicate an early infection or contamination by virus and increased protein levels of these cytokines show a later pathogenesis condition. Therefore, the current invention discloses a method to detect pathogen infection, especially virus infection. The method comprises taking the body fluid from a subject, preferably the blood sample, test at least one or more cytokines level within the sample, and the elevated cytokines level (compared with normal subject) suggest the presence of potential pathogen especially virus infection. The suitable cytokines are selected from the group of IFNa, IFNg, interleukin-5, 8, 15, 18 and inducible protein 10 (IP 10). The elevated cytokines levels indicate the subject has a high possibility of suffering virus infection. Preferably multiple cytokines are used as marker during the test and any one of them having an increased level is an indicator of possible pathogen (most likely virus) infection. The cytokine level can be determined either from their protein level or their mRNA level in the blood sample.

Well known methods such as ELISA or PCR can be used. Instead of other virus infection detection method, the current invention does not rely on the information from the virus, in another word; this method can tell the subject is likely having a virus infection even the virus is unknown. Therefore, one important application of current method is to use this method as a health examination tool, if the elevated cytokine level is found, further examination can be conducted to identify the cause. Another important application of current method is to use this method as a blood product contamination test, if the elevated cytokine level is found in the blood product; it should not be used to avoid the potential transmission of pathogens from the donor to the receiving patients.

Another aspect of the current inventions disclose methods to remove the anti coagulation agent in the blood to treat its side effect. When performing blood purification such as blood dialysis, anti coagulation agents are widely used to prevent blood coagulation in the blood purification system during the blood purification procedure. The most widely used anti coagulation agent is heparin. To prevent its side effect such as causing bleeding, the amount used has to be carefully monitored and adjusted accordingly. Protamine is used to neutralize the heparin after the dialysis. This also causes other side effects such as allergy reaction. The current invention discloses an anti coagulation agent removal device. It can remove the heparin or other anti coagulation agents in the blood by performing blood purification. It utilizes a vessel similar to the blood purification device previously described in hemoperfusion, in which solid phase immobilized with functional groups/molecules having affinity to anti coagulation agents is placed inside. For example, in order to remove heparin, Solid phase immobilized with functional groups/molecules having affinity to heparin is used. When blood pass through the vessel, the heparin will absorb to the solid phase inside therefore be removed from the blood and the clean blood will go back to the patient without causing side effect of the heparin. The affinity molecule can be antibody or aptamer for the target anti coagulation agent, e.g. antibody specific to warfarin. They can also be any other type of affinity molecules such as molecular imprinting polymer or lectin. They can be a single molecule or mixtures. One suitable type of affinity molecule for heparin is cationic molecule especially the poly cationic molecule, such as molecules having multiple amine groups or phosphonium groups. Examples of them include lysine, arginine, spermine, polylysine, polyethylenimine or the like. The amine group can be primary amine, secondary amine, tertiary amine or quaternary amine. These are many well known methods to couple them to the solid phase. For example, the amine containing molecule can couple to the carboxylic acid containing solid phase by forming amide bond using EDC type coupling reagent. Or the solid phase itself can have affinity to the heparin. For example the solid phase having multiple cationic groups (e.g. cross linked polyethylenimine spheres, anion exchange resins) can be used to capture heparin. There are many anion exchange resins commercially available. The heparin has higher affinity to anion exchange resins than other components in the blood. Therefore a small amount of anion exchange resin can efficiently remove the heparin from the blood. In some embodiment, the blood passes through hollow fibers. The hollow fiber is immobilized with heparin affinity molecule such as poly amine. If the whole blood including the blood cell is passing through the solid phase, the solid phase can be coated with a layer of thin film or biocompatible polymers to increase the bio compatibility of the solid phase with the blood cells. Examples of these biocompatible film or polymer include albumin, glutin, agar, acrylic acid gel, PEG, PVA and etc. In one example, 1 mm diameter polystyrene particle having amine groups is used as solid phase adsorbent for heparin. They are placed in a vessel with filters at the two ends connecting to the blood vessels. The pore of filter is smaller than the polystyrene particle but bigger than the blood cells therefore allow blood cells passing through the vessel freely but not allow the particles escape. After the blood pass through, the heparin will be captured by the solid phase and the clean blood will return to the patient. The solid phase can be equilibrated with electrolyte similar to plasma to prevent it interrupt the blood electrolyte composition. In other embodiments, the blood is withdraw from the patient and divided into blood cells component and plasma component using varieties of means such as plasma separator. The plasma pass through the solid phase adsorbent to remove the heparin and then return to the patient or combine with the bloods cells then return to the patient. In some examples, the vessel contains many hollow fibers used for plasma separation and these fibers are surrounded by solid phase adsorbent inside the vessel. The whole blood pass through the hollow fiber so the blood cell will not escape from the fiber but the heparin containing plasma will cross the fiber membrane and contact the solid phase adsorbent therefore the heparin is removed. In one example, polysulfone membrane is used to make the plasma separation hollow fiber with the average pore size of 0.2-0.6 um and 0.5 m² total membrane area. The vessel is filled with 50 g D201 or D301 type weak basic poly styrene anion exchange resin outside the fiber. The blood from artery enter the vessel from one end (blood inlet) and pass through the fiber and then go out from another end of the vessel (blood outlet) returning to the vein of the patient. Because the blood cells do not contact the resin directly, biocompatibility is assured. The heparin removal device can be connected to other blood purification device (e.g. a dialysis column) in a tandem format. Therefore the blood passes through the dialyzer and then flow into the heparin removal vessel to remove the heparin in the blood. Next the blood is returned to the patient. The heparin can be added in the tubing before the dialyzer. The heparin removal device can also be integrated into dialyzer or other blood purification device by place the heparin removal solid phase at the blood outlet end of the dialyzer/blood purification device. For example in a dialyzer, the portion of hollow fiber close to the blood outlet end can be coated with polyamines to remove the heparin.

Another aspect of the current inventions relate to methods to treat sepsis especially sepsis shock. The death of sepsis shock is mainly due to the released bacterial endotoxin causing the deadly immune response. Therefore the current invention use blood purification to remove the endotoxin from blood to treat it, the bacteria themselves and the immunifactor increasing sepsis condition (e.g. IL-6, IL-8, TNF) can also be removed as well. Hemopurifier and blood dialysis device can be coated with affinity ligand for endotoxin (e.g. antibodies specific for the endotoxin, Polymyxin B) to remove the endotoxin from blood. They can also be coated with affinity ligands (e.g. antibodies) targeting bacterial themselves and the IL-6, IL-8, TNF like immunifactor to remove them from blood as well as affinity ligand for bacterial endotoxin. Blood purification allows many different kinds of affinity groups targeting multiple targets (e.g. endotoxin from different bacteria) can be used in combination easily and it has minimal side effect. Also, it is possible that the currently used blood dialysis device used for kidney problem and patients suffering immune problems can also be used to treat sepsis since these devices can also remove small molecules and immune molecules, however the removal is none specific therefore can has more side effects.

The methods described in the current inventions all utilize blood purification techniques therefore the protocols and devices used in different applications can be readily adopted or modified from each other and some of them can be used interchangeable by a skilled in the art.

Cancer/tumor cells (including the cancer stem cell) in the circulating blood can cause tumor metastasis, which will generate secondary tumor/cancer. Surgical operation can also cause the release of more tumor cells to the blood. Therefore, a method that can remove the cancer cells from blood will help the treatment of cancer, especially in reducing the risk of tumor metastasis.

The current invention provide methods to treat cancer especially to prevent tumor metastasis and tumor recurrence by removing and/or inactivating (e.g. killing) the circulating tumor cells (CTC) in the blood after removing the tumor or treating the tumor with therapeutical means such as surgery, chemotherapy, radiation therapy, photodynamic therapy, photon radiation therapy, laser therapy, microwave therapy, cryogenic therapy, heat therapy or combinations of them. In some embodiments, the therapeutical means targets the primary tumor. The method to prevent tumor metastasis and tumor recurrence in the current invention comprises two steps 1) removing the tumor or treating the tumor with therapeutical means such as surgery, chemotherapy, radiation therapy, photodynamic therapy, photon radiation therapy, laser therapy, microwave therapy, cryogenic therapy, heat therapy or combinations of them; next 2) removing the circulating tumor cells from the blood and/or inactivating the circulating tumor cells by extracorporeally circulating blood. It also provides a method to measure the amount of circulating tumor cells.

In general, these circulating tumor cells are removed (inactivated) by blood purification (e.g. hemopurification) of extracorporeally circulating blood through a blood purifier that can remove/kill the circulating tumor cells in the blood and/or inactivate the CTC while it is outside the body by extracorporeally circulating blood. What passes the blood purifier or what is treated with CTC inactivation means can be either whole blood or the blood component containing the CTC. Hemopurifier and blood dialysis device are widely used for many disease such as kidney failure. A solid phase adsorbent that has affinity to the tumor cells can be placed in the blood purifier for the blood purification. For example, the solid phase adsorbent (e.g. column, filter, fiber, membrane, particle) coated with affinity molecules that can selectively bind with the tumor cells can be used in the blood purification device to remove these cells. Preferably, these affinity molecules have no or low affinity to majority of other normal blood cells.

Because the tumor cells are immobilized on the solid phase adsorbent during the blood purification, the cells on it can be counted after the tumor cell removal operation to provide an accurate measurement of the numbers and types of circulating tumor cells in the patient, which can be used to evaluate the anti tumor treatment and guide the future treatment. For example, detergent can be added to the solid phase to lyse the cell and the lysate can be tested with PCR or ELISA to measure the amount of tumor cells in it. The tumor cells can also be eluted from the solid phase using elution buffer (e.g. low pH glycine solution) and the eluted cells can be collected for diagnosis and further cultured for varieties of applications.

Using affinity molecules coupled with anti cancer drug is widely used in the treatment of tumor. One can readily adopt the method and principle of them for the current invention to make affinity CTC adsorbent. Examples of the affinity molecules include cancer cell specific antibodies, small molecules having specific affinity to the cancer cell surface markers, aptamer specific to the tumor cell surfaces and etc. For example, antibody to the unhealthy white blood cells can be coated to the column in the blood purifier to treat leukemia. Antibody to the lung cancer cells can be coated to the column in the blood purifier to treat lung cancer. The blood purification step can be performed in combination with chemotherapy. In preferred embodiments, it can also be performed after the tumor removing surgery to eliminate the released tumor cells. The solid phase adsorbent is immobilized with affinity ligand to the surface marker of tumor cell and/or epidermal cell/epithelium, such as antibody or aptamer specific to Cytoketatin and/or EPCAM (Epithelial specific antigen). The affinity ligand can also be specific to certain type of tumor cell such as using antibody to prostate-specific membrane antigen for prostate cancer. The affinity ligand can be protein, nucleic acid as well as molecular imprinting polymers, small molecules and etc. It can be single molecules or mixture of different affinity molecules. Because the solid phase support (adsorbent) has affinity ligand on it to the tumor cell or itself has affinity to tumor cells, extracorporeally circulating blood through the solid phase will remove the tumor cells from the blood. The affinity ligand can also be specific to other tumor cell marker such as HER-2 (HER-2/neu), EGFR, mammanaglobin protein, PMSA, EpCAM, GA733-2 and MUC1. One can readily find many suitable tumor cell surface markers from the literature.

The surface marker of tumor cell can also be introduced artificially. The principle is described as following: the tumor cell has its endogenous marker A; affinity molecule B that can bind with A is conjugated with maker C, when it is mixed with tumor cell, the tumor cell will have marker Con its surface. The solid phase having affinity to C will be used to remove tumor cell. For example, biotin (marker C) labeled EPCAM antibody (affinity molecule B) is applied to the blood containing tumor cell, avidin or streptavidin coated solid phase will be used to remove tumor cell, which mechanism is similar to those of the CellPro Ceprate SC Stem Cell system. The biotin labeled EPCAM antibody can be either injected to the patient or mixed with the blood after the blood is drawn from the patient. Affinity molecule B can be either a single species of molecule or mixtures of different affinity molecules. In some embodiments, C and B can be the same molecule. For example, it could be EPCAM antibody generated from goat. The solid phase can be coated with anti goat IgG antibody generated from rabbit. In another word, affinity molecule B itself is the marker C.

The solid phase can also be coated with anti coagulation agent such as heparin to prevent blood coagulation. Blocking factor such as blocking antibody, solubler tumor antigen and their antibody-antigen complex as well as the immune suppressive microvesicular particles described in US patent application 20090304677 can inhibit the immune function against tumor. Other immune inhibiting substance include IL-10, TGF-β, VEG, PGE2, Fas ligand, MHC I, MHC II, CD44, placental alkaline phosphatase, TSG-101, MHC I-peptide complexes, MHC II-peptide complexes. Therefore, affinity molecule (e.g. antibody, lectin, aptamer) for them can also coated on the solid phase to remove them from the blood to boost the immune function of the patient against tumor.

Some of the tumor cells in the blood are coated with antibody of the patient, which may prevent them from being captured with solid phase support described above. Therefore, affinity molecules for antigen-antibody complex can be coated on the solid phase to capture the antibody bound tumor cell. For example, complement molecule such as C1q can be coated on the solid phase to capture this type of tumor cells. Other C1q type molecules or molecules have the similar function as C1q can also be used instead, which are described above in the pathogen removal treatment. The tumor cell surface antigen can also be coated on the solid phase to capture the antibody bound tumor cells since the antibody on it has two binding sites. The plasma can also be separated from the blood and only allow the cell components passing the solid phase adsorbent, so the affinity molecule for antibody (e.g. protein A) can be coated on the solid phase to capture the tumor cell without capture the soluble antibody in plasma. None specific column such as TR350 or PH350 can also be used.

The solid phase for CTC removal can be of a column shape or packed into a column shape with particles or fibers. It can also be membrane, filter, fiber, hollow fiber, tube, micro particle, magnetic particle or other format, as long as is has suitable surface property to couple with affinity molecule. Two preferred type of solid phase adsorbent coated with affinity ligand are particles and fibers. The fiber can be made into mesh or textile having suitable pore size (e.g. 10˜150 um). For example, one can use the similar fiber in Toraymyxin PMX-20R device. Toraymyxin PMX-20R is an extracorporeal hemoperfusion device which is composed of polymyxin B covalently immobilized polystyrene derived fibers. The cartridge is 225 mm×63 mm in size containing 56 g dry fiber. For CTC removal application one can coat the polystyrene derived fibers with affinity ligand for CTC instead of using polymyxin. The polymyxin B coated fiber used in the polymyxin B itself can also be used to directly couple with the affinity ligand for CTC since the coated polymyxin B still has several free amines for the coupling. Other types of fiber can also be used such as cellulose fiber, polysulfone fiber, polyethersulfone fiber, polyvinyl alcohol fiber, cellulose acetate fiber, polyethylene fiber, polypropylene fiber, polymethylmethacrylate fiber, polyacrylonitrile fiber, cellulose triacetate fiber or the combination/conjugation of them. These fibers can be readily derivatized to couple with affinity ligands. The fiber can also be made into mesh or textile having suitable pore size (e.g. 10˜250 um) therefore can optionally function as a filter or relative obstacle for CTC to help the affinity capture of CTC. When particles are used, bigger particle (e.g. >50 um) has much larger size than blood cell therefore can be blocked from entering into patient by using suitable filter (e.g. pore size around 30 or 40 um) which allows blood cells pass through freely. Smaller particles have larger surface area in favor of absorption but too small size (e.g. <10 um) will make it difficult to separate from blood cells using filters. Using magnetic micro particle allows smaller particle be used by applying magnetic field to remove magnetic particle to prevent them entering patient's body. When magnetic particle is used to remove CTC in the blood component (e.g. white blood cell portion containing CTC), the blood component can be mixed with magnetic particle in a container instead of passing though a blood purification cartridge. The procedure of using magnetic particle to isolate cell is well known to the skilled in the art. Similarly, the non magnetic micro particle having affinity ligand to the CTC can also be mixed with the CTC containing blood component and then separate the rest of cells from the particle with suitable pore size filter to prevent the micro particle going to the body with the rest of cells (e.g. using 50 um size micro particle with 30 um pore size filter).

The solid phase coated with different affinity molecules (e.g. different antibodies) can also be combined in the blood purification to remove CTC. They can be a mixture of several different solid phase adsorbents each having their unique affinity molecules or simply immobilizing several types of affinity molecules on the same solid phase support. One can also use several different type of CTC removing cartridge in serial to reach maximal CTC removing effect.

In some embodiments, the blood passes through the hollow fiber. The membrane of the hollow fiber is coated with tumor cell affinity ligand. The surface of hollow fiber can be chemically modified (e.g. crafting or polymerization) to introduce functional group (e.g. amine or carboxyl group) to couple with affinity molecules. Hollow fiber is widely used in dialysis, plasma separation and blood perfusion.

In some embodiments, the blood is withdrawn from the patient and then passes through the solid phase adsorbent particles having affinity to tumor cells in a cartridge. In other embodiments, the blood is withdraw from the patient and divided into blood cells component and plasma component using varieties of means such as plasma separator. The cellular component passes through the solid phase adsorbent (e.g. particles, membranes, filters and etc.) to remove the tumor cell and then return to the patient or combine with the plasma then return to the patient.

The withdrawn whole blood can be first separated into to several components by using blood cell separation means such as apheresis. Only the part containing a great number of CTC will receive the CTC removal/inactivation treatment. For example, in order to separate the CTC containing blood component from the whole blood, many methods can be applied such as leukapheresis, size based filtration, centrifuge and elutriation. Many blood cell separation devices can be used such as varieties of blood cell separator, e.g. cs3000plus blood cell separator, COBEVR Spectra system and the Elutra system (Caridian BCT). For example, one can also use a hollow fiber plasma separator type device to separator the CTC from other blood cells. The membrane of the hollow fibers have larger pore than those used in plasma separation. The pore is big enough to allow red blood cell and platelet to pass but smaller than the size of significant portion of CTC (e.g. 8 um, 10 um, 12 um or 15 um). After the whole blood pass, the inside especially the end part of the hollow fiber will be enriched with CTC and the liquid outside the hollow fiber will contain red blood cell, some white blood cell, plasma and platelet which can be send back to the patient directly. Optionally, the solid phase adsorbent (particle size>pore size) coated with affinity ligand for CTC can be filled in the hollow fiber. The membrane of the hollow fiber can also be coated with affinity ligand as well. Therefore it will also function as a blood purifier. And if this kind of configuration is used, in some applications the pore size can also be bigger than the CTC (e.g. 20 um˜50 um) and outside the hollow fiber can also be filled with CTC adsorbent.

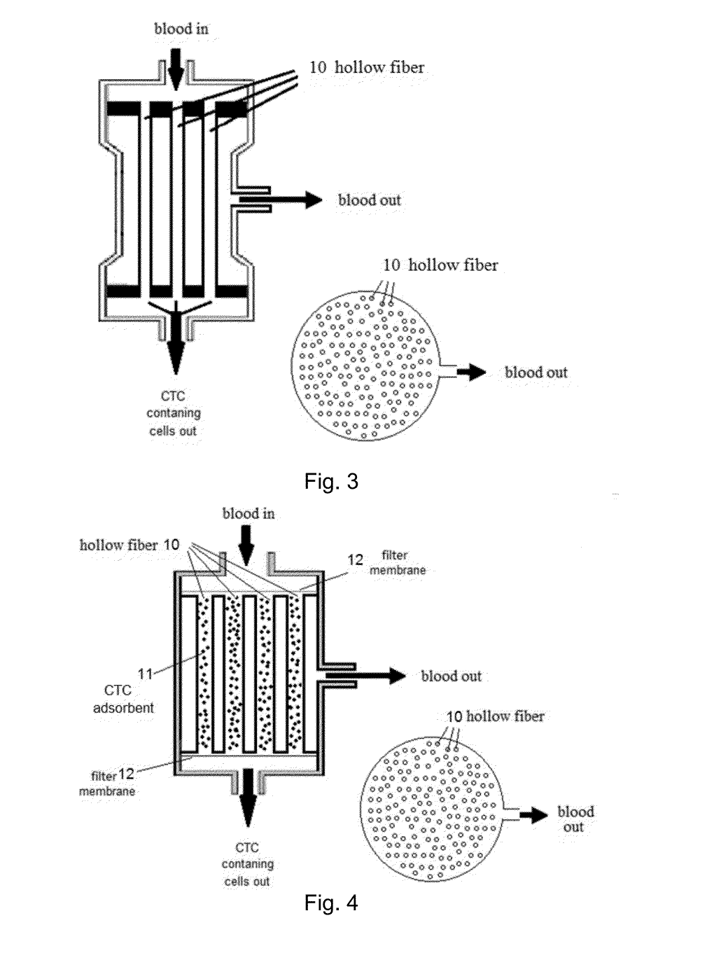

In some embodiments, the blood is withdrawn from the patient and extracorporeal circulating is established. As in example 5, the blood passes through a cartridge described in FIG. 3. The cartridge contains many hollow fibers 10 made of polysulfone membrane. Suitable diameter of the fiber can be selected from 100 um to 1000 um. The total area of the hollow fiber membrane is 2 m² and the pore size of the membrane is 12 um. One end of the cartridge has blood inlet to connect with the blood from artery and the cartridge also has blood outlet to return blood to the vein. Optionally, inside the hollow fiber 10 is filled with solid phase CTC adsorbent 11 in the shape of particles or fibers (size>the pore size of the membrane 12 and the pore size of hollow fiber membrane, for example, particle size is 100 um and the filter membrane 12 pore size is 30 um) having affinity to the CTC as shown in FIG. 4. The left parts of FIGS. 3 and 4 show a schematic illustration of a longitudinal cross section of the device and the right parts of FIGS. 3 and 4 show a schematic illustration of a horizontal cross section of the device. Another end of the cartridge in FIG. 3 or FIG. 4 has a CTC containing cell out outlet, which can have a valve to control the on/off/speed of the flow. The blood in path can also have a valve to adjust the on/off and flow rate of the blood flow as well as to provide other liquid such as cartridge washing liquid to the cartridge. When the blood pass through the cartridge, the red blood cell, platelet, plasma and some white blood cell will pass the wall of the hollow fiber and exit from the cartridge from the blood out outlet and then go back to the patient. The CTC and some white blood cell/plasma will remain in the hollow fiber and exit the cartridge from the CTC containing cell out outlet when the valve is open. When smaller pore size (e.g. 10 um) hollow fiber is used, most CTC will be retained but more white blood cell will also be retained. When larger pore (e.g. 20 um) hollow fiber is used, less white blood cell will be retained but some CTC may also escape. The optimal pore size can be selected based on the size of the CTC from the patient by analyzing the CTC from the patient's blood sample. The hollow fiber can also be made with other type of synthetic polymers or inorganic materials besides polysulfone membrane. The pore forming/fiber wall crossing channel can have the same diameter in the inner/outer sides of the hollow fiber 10 or have different size in the inner/outer sides of the hollow fiber 10. For example, the pore size at the inner wall of the hollow fiber can be bigger than the pore size at the outer wall. So the diameter of channel across the hollow fiber wall shrinks from the inside of the hollow fiber to the outside of the hollow fiber. The inside pore size can be even bigger than the size of CTC (e.g. 30˜50 um). The valve can be always open or open periodically or only open at the end of the procedure to drain the CTC containing cells. Additional liquid can be added from the blood in end to help to drain the cells. The valve can also be kept close all the time and the cartridge is discarded at the end in some cases. The effluent CTC containing cells can then be treated with other CTC removal/inactivating means. For example, it can pass through a cartridge containing CTC affinity adsorbent before going back to the patient. It can also pass another same type cartridge to further remove the blood cells (which can be sent back to the patient) from the CTC containing portion.

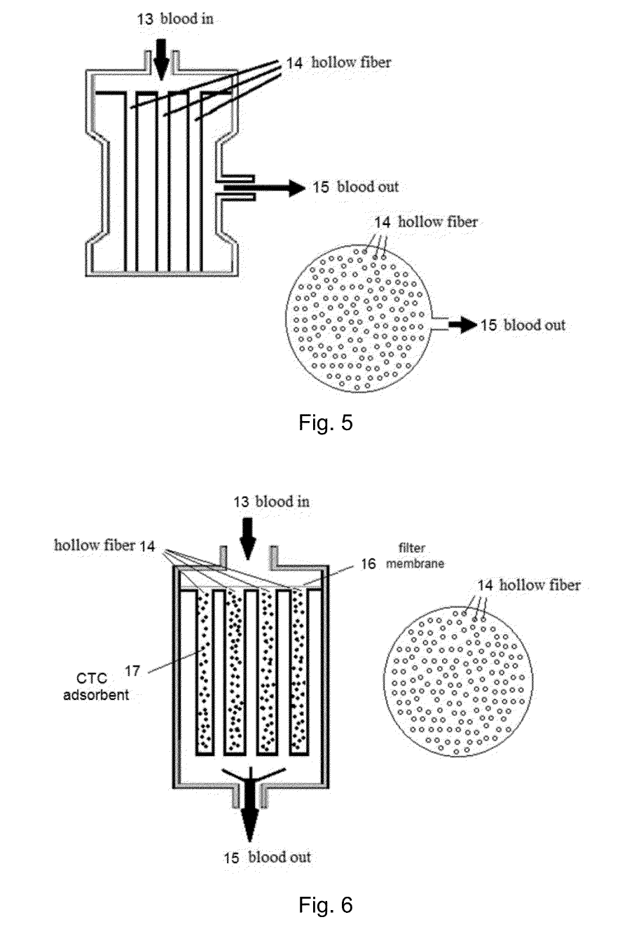

In example 6, the blood passes through a cartridge as described in FIG. 5. The cartridge contains many hollow fibers 14 made of polysulfone membrane. Suitable diameter of the fiber can be selected from 100 um to 1000 um. In one example, the diameter is 300 um. The total area of the membrane is 5 m² and the pore size of the membrane is 15 um. One end of the cartridge has blood inlet 13 to connect with the blood from artery and the cartridge also has blood outlet 15 to return blood to the vein. Optionally, inside the hollow fiber 14 is filled with adsorbent solid phase materials such as particles or fibers (size>the pore size of the hollow fiber membrane and the pore size of filter membrane 16, for example, particle size is um and the filter membrane 16 pore size is 50 um) having affinity to the CTC as shown in FIG. 6. The left part of FIGS. 5 and 6 show a schematic illustration of a longitudinal cross section of the devices and the right part of FIGS. 5 and 6 shows a schematic illustration of a horizontal cross section of the devices. Unlike the devices in FIGS. 3 and 4, the cartridge has no CTC containing cell out outlet in another end. The other end of the hollow fiber is sealed. When the blood pass through the cartridge, the red blood cell, platelet, plasma and some white blood cell will pass the wall of the hollow fiber and exit from the cartridge from the blood out outlet 15 and then go back to the patient. The CTC and some white blood cell/plasma will remain in the hollow fiber and will not exit the cartridge. When smaller pore size (e.g. 10 um) hollow fiber is used, most CTC will be retained but more white blood cell will also be retained. When larger pore (e.g. 20 um) hollow fiber is used, less white blood cell will also be retained but some CTC may also escape. The optimal pore size can be selected based on the size of the CTC from the patient by analyzing the CTC from the patient's blood sample. The hollow fiber can also be made with other type of synthetic polymers or inorganic materials besides polysulfone membrane. The pore forming/fiber wall crossing channel can have the same diameter in the inner/outer sides of the hollow fiber or different size in the inner/outer sides of the hollow fiber. For example, the pore size at the inner wall of the hollow fiber can be bigger than the pore size at the outer wall. So the diameter of channel across the hollow fiber wall shrinks from the inside of the hollow fiber to the outside of the hollow fiber. The inside pore size can be even bigger than the size of CTC (e.g. 30˜50 um). It is similar to the device in FIGS. 3 and 4 that keeps the CTC out valve close all the time. The CTC containing cells in the hollow fiber can be eluted out from the blood in outlet or discarded. If desired, the effluent CTC containing cells can be treated with other CTC removal/inactivating means and then return to the patient. The devices described in FIG. 4, 6 are similar to those in FIG. 3, 5 with additional CTC adsorbent inside the hollow fiber. Filter membranes are placed inside the cartridge to prevent the CTC adsorbent going into the patient. The pore of the filter membrane is bigger than the size of cells but smaller than the size of the CTC adsorbent.

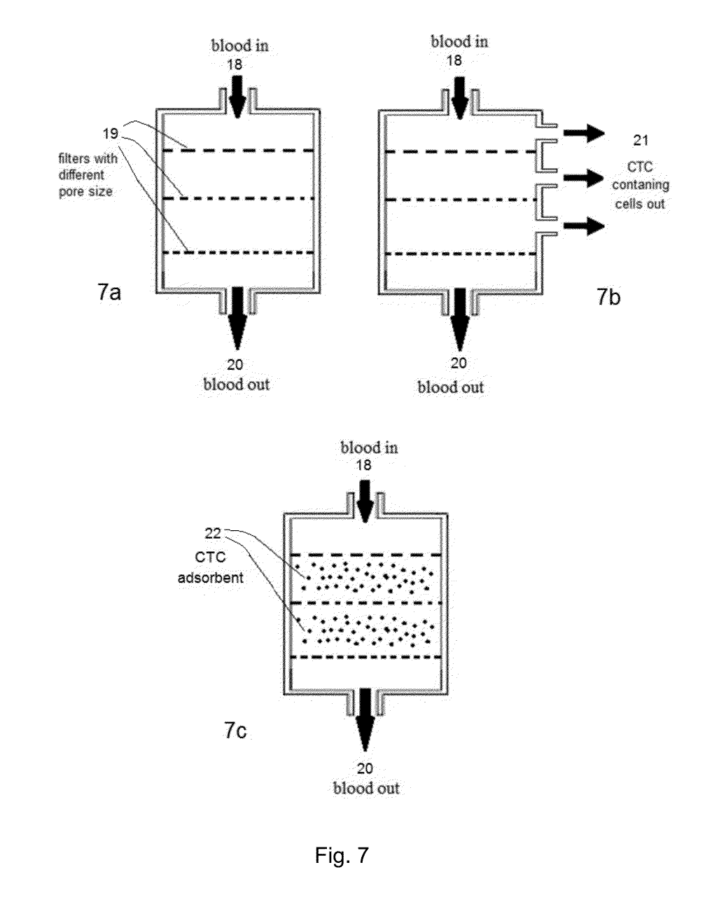

FIG. 7 shows examples of another type of CTC removal device. The device has multiple filter membranes or plates 19 with different pore size inside the cartridge. The pore of the filters close to the blood in inlet 18 is bigger than the pore size of the filter close to the blood out outlet 20. For example as shown in FIG. 7a, the first filter (top one) has pore size of 35 um, the second filter (middle one) has pore size of 20 um and the third (bottom one) has pore size of 12 um. In another example, the pore size changes from 30 um to 15 um to 8 um. There can be outlet 21 above and between the filters as shown in FIG. 7b to drain the CTC containing cells for further processing (e.g. to couple with other CTC removal/inactivating means). CTC adsorbent 22 can also be filled in the cartridge as shown in FIG. 7c. In one example, the filters 19 are polysulfone membrane filters each having surface area of 0.1 m² with 45 um, 30 um and 20 um pore size respectively.

In some cases the CTC removal cartridge contains only one layer of filter to remove the CTC. In this kind of cartridge, the cell outlet above the filter can be open periodically to drain the accumulated CTC containing cells on top of the filter, which may pile up and block the pore of the filter therefore affect the blood flow going through.

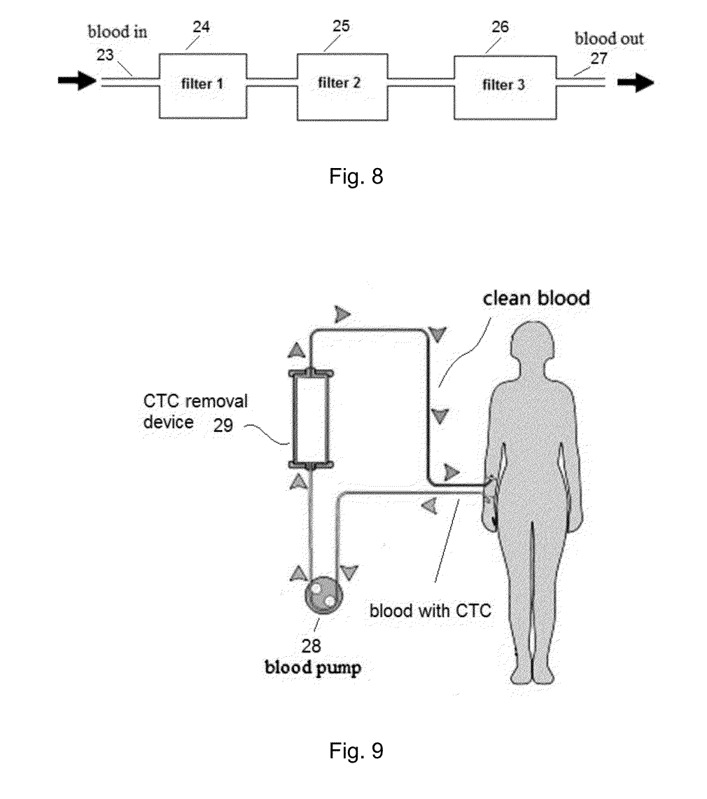

In example 7, the cartridge having filters with different pore size is placed sequentially and each cartridge only has one size filter inside. As shown in FIG. 8, three filters having different pore size is placed in the extracorporeal circulating blood path. Each filter has a filtration membrane of 0.05 m² surface area. Filter 24 has a pore size of 30 um, filter 25 has a pore size of 20 um and filter 26 has a pore size of 12 um. The blood inlet 23 is connected with filter 24 and the blood out outlet 27 is connected with filter 26. There can be cell outlet in each cartridge (before the filter) to drain the retained cells for further processing. Similarly, the devices described in this patent can also be used in combination by placing them sequentially in the blood path.

Another method to remove CTC is to use blood cell separator. When the blood is processed with blood cell separator, most CTC will stay within the leukocyte component in most cases. In some cases CTC will be in the mononuclear cells component and in some cases the CTC will stay in the monocyte portion depending on the cell separator type, its parameter and the nature of the CTC cells (the exact distribution of CTC can be determined experimentally by testing a small amount of blood from the patient). One can readily isolate these components using blood cell separator. Next the portion containing the CTC (e.g. the monocyte portion or the mononuclear cell portion or the entire leukocyte portion) is given the CTC removal/inactivation treatment either continually or in a batch format. Other blood components can be sent back to the body directly after the separation or be combined with the blood component being treated then return to the body. Optionally the other blood components can also pass a different blood purifier or CTC inactivating means before go back to the body. The CTC containing leucocytes can also be treated with centrifugation based device again (and optionally be added with buffer/liquid) to further enrich the CTC and remove the healthy cell (e.g. platelet) before go to the next treatment.

In some embodiments, the blood or blood component passing through blood purifier is repeated a few times during the treatment. For example, after the blood or blood component passing through a cartridge filled with adsorbent it is re introduced to the cartridge to allow it pass the adsorbent again before going back to the patient.

The current invention described several methods/devices to remove/inactivate CTC. These means can be used independently or in any combination if they are compatible as well as be repeated in one treatment session. For example, the whole blood can first be treated with a centrifugation type blood cell separator and the CTC containing leucocytes is sent to an affinity capture adsorbent based purifier or a filtration based separator. After filtration the blocked CTC/other cells (e.g. leucocytes) can be discarded or pass through an affinity capture based purifier or a CTC inactivating device before return to the patient. In another example, the whole blood first pass through a filtration type CTC removing device and the blocked CTC/other cells then pass through an affinity capture based purifier or a CTC inactivating device (or being treated with CTC inactivating means) before return to the patient. In a third example, the whole blood first passes through a filtration type CTC removing device and the blocked CTC/other cells then are sent to a centrifugation type blood cell separator. The resulting enriched CTC containing component can be discarded or be further treated with other type CTC removing/inactivating device/devices/means before return to the patient. At any stage, the resulting blood component containing no or only small number of CTC can be send back to the patient or optionally be treated with another type of CTC removing/inactivating device/means before return to the patient if this small number of CTC also need to be removed.

The CTC removal/inactivation treatment can be performed either in continuous flow fashion or intermittent flow fashion. The blood withdrawn/return and/or blood component separation can also be done in either continuous flow fashion or intermittent flow fashion. For example, because different cells have different density, one can use centrifugation based blood cell separator to separator the CTC containing blood component. After centrifugation of the whole blood, the red blood cell is at the bottom and the white blood cell and CTC is on top of it. On top of the white blood cell and CTC is plasma. By adjusting the centrifugation parameter, minimal red blood cell and platelet stay in the white blood cell/CTC layer. The red blood cell and plasma can be sent back to the patient continuously during the separation by tubing inside these layers. The CTC containing portion can be sent to the CTC removing/inactivating device (or being treated with CTC inactivating means) continuously or be sent to the CTC removing/inactivating device (or being treated with CTC inactivating means) at the end of the separation or intermittently when it reach certain volume. The centrifugation can also be in a continuous flow centrifugation fashion or intermittent flow centrifugation fashion, both of which are widely used in apheresis. The CTC removing/inactivating process can also be done in an intermittent flow fashion (e.g. batch format) to ensure sufficient treatment time. This strategy is described previously in the pathogen removal/inactivation methods. For example, the blood can be withdrawn continuously and optionally separated into blood components continuously and pass the blood purifier and return to the patient continuously; while in another example, certain volume of blood/blood component is withdrawn and been treated for certain period of time then return to the patient and then the next batch of blood/blood component is withdrawn for treatment. In another example, the blood going to the blood cell separator is done continuously but the blood component having CTC going to the CTC removal/inactivating device (or being treated with CTC inactivating means) for treatment and treated part returning to the patient are done in batch.

Because the treatments in the current invention can cause some blood components being lost or inactivated, additional blood components can be given to the patient during or after the treatments. For example, the patient can be given suitable amount of red blood cell or platelet from the healthy donor if they are lost or killed. The patients can also be given leucocytes if needed. The leucocytes can be from healthy donor or from the patient' own resource, e.g. cultured from patient's bone marrow or stem cells. Medicines can boost the production of blood cells can also be given before or after the treatment. The amount of these blood components in the patient can be monitored to guide the infusion. Liquid/buffer (e.g. artificial plasma) can also be added to the CTC containing blood component after the apheresis to aid the CTC removing/inactivating. Liquid/buffer (e.g. artificial plasma) can also be added to blood to aid the apheresis.

In some embodiments, the CTC containing white blood cells (e.g. those from centrifuge or filtration device) can simply be discarded without further treatment. Either all of them or portion of them can be discarded. External white blood cells (e.g. from donor or culture) and/or other blood component can be given to the patient to compensate the lost white blood cells and/or other blood components. After a few days the treatment can be repeated until the CTC reach desired level. In some applications it is preferred that each treatment need to remove more than ⅓ of the total CTC in the blood, which is determined by the separation efficacy, treatment time and the volume of cells discarded.

Alternatively, all or certain volume of the whole blood from the patient can be withdrawn and discarded and the resulting blood lost is compensated with blood infusion from healthy donor or culture of own bone marrow/stem cells. The treatment can be repeated several times (e.g. once every 3 days or once a week) until the CTC reduce to desired level. In some applications it is preferred that each treatment need to remove more than ⅕ of the blood from the patient. For example, in each treatment 1˜2 litter of blood is withdrawn/discarded and at least the same amount of healthy blood from donor is infused at the same time or right after the blood removal.

Means that can inactivate (e.g. kill) the CTC can also be applied to the extracorporeally circulating blood or CTC containing blood component so the systematic side effect is minimized due to only the blood outside the body/isolated blood component part is treated.

In some embodiments, heating is used to inactivate CTC. The blood is withdrawn and then is heated to be higher than 40 degree (e.g. 42˜48 degree). Varieties of heating means can be applied such as passing through a heat exchanger or be microwave treated or be IR radiated. It can be either a continuous flow fashion or an intermittent flow fashion to ensure sufficient heating time. The heated blood is then returned to the body. In some embodiments the temperature need to be controlled (e.g. cooled) before the returning to avoid the body temperature of the patient rise too high (e.g. above 40 degree). Cooling means such as ice pad can be applied to the patient's neck and head to avoid high temperature to the CNS. Alternatively, the blood is withdrawn and passes through a blood cell separator (e.g. centrifugation based or filtration based). Only the CTC rich part (e.g. the leukocyte portion from centrifugation) is treated with heat before return to the body while other parts are returned to the patient directly. In one example, the CTC containing leukocyte potion is heated for 30 min at 42 degree and then send back to the body. Other temperature and heating time can also be applied as long as the CTC can be killed. Preferably the temperature and heating time selected should cause minimal normal blood cell damage. Additional treatments such as CTC removal with affinity adsorbent can also be applied before the blood/blood component go back to the patient.

In some embodiments, UV is used to inactivate CTC. Either extracorporeally circulating whole blood or the blood component rich of CTC (e.g. the leukocyte portion from centrifugation means such as blood cell separator) can be irradiated when they are outside the body. One preferred wavelength is those at which nucleic acid has strong absorbance, e.g. 250-260 nm. The radiation intensity and time should be enough to kill the CTC and preferably cause less damage to the normal blood cell (e.g. 10 J/ml). The extracorporeally circulating and/or UV treatment can be either in a continuous flow fashion or an intermittent flow fashion to ensure sufficient irradiation time. The UV treatment condition can be determined experimentally by test a small amount of blood containing CTC first. Additional treatments such as CTC removal with affinity adsorbent can also be applied before the blood/blood component go back to the patient.

In some embodiments, chemical agent (e.g. antitumor drug) is used to inactivate CTC. Either extracorporeally circulating whole blood or the isolated blood component rich of CTC (e.g. the leukocyte portion from centrifugation) can be treated with CTC inactivating agents while they are outside the body. Anti tumor agent/drug especially those directly kill/inactivate cancer cell can be used. Examples of suitable agents/drugs include but not limited to alkylating agents (e.g. phosphoramide mustard, thio-TEPA, nitrosoureas type drug such as carmustine, lomustine, semustine and streptozotocin), bleomycin, adriamycin, mitomycin, cisplatin, [PtCl(H₂O)(NH₃)₂]+ and taxol. If photon such as IR, visible light or UV is provided to the blood/blood component outside the body, photoactive agents (e.g. those used to treat blood products) such as phenothiazine dyes, methylene blue, vitamin B2, 1595 psoralen (e.g. 8-MOP, AMT), agents used in photodynamic therapy such as photosensitizers (e.g. Photofrin or Levulan or nano particle TiO2) can also be used to inactivate the CTC. These photoactive agents/photosensitizers can also be coupled with affinity ligand to the CTC to provide better selectivity. Preferably they (either one CTC inactivating agent or the combination of several agents) are added to the CTC rich blood component or to the whole blood after the blood is withdrawn from the patient to avoid these drugs cause side effect inside the body. The amount of the agent used should be sufficient to inactivate the CTC during the treatment, which can be found from literatures (e.g. 10 times the reported IC 50) or determined experimentally easily. Optionally these agents are removed or inactivated (e.g. neutralized) from the blood/blood component after the inactivating treatment (e.g. mixing and incubating with these agents, photon irradiation) but before the blood/blood component is returned to the patient to reduce the potential side effect of these agents to the patient, except for certain agent that lose activity before the blood going back (e.g. short half life). For example, by passing the blood/blood component through a blood purification device (such as a hemo perfusion column filled with 100 g adsorbent) filled with adsorbent (e.g. charcoal, adsorption resin) that can absorb these agents or a blood dialyzer that can remove drugs from the blood. There are many these types of devices and techniques available for blood purification/blood perfusion/blood dialysis to remove drugs in the blood. One can readily adopt them for the current application. For example, crosslinked agar entrapping attapulgite clay, Pall MB1 filter, Maco Pharma Blueflex filter or LeucoVir MB filter can be used to remove methylene blue in the blood or blood component. A blood dialyzer using half permeable membrane or filter membrane can also selectively remove the anti tumor agent from the blood or blood component because of the difference in their molecular weight or size. The adsorbent filled blood purification device can also be used to remove these anti tumor agent when the blood or blood component pass through the device. The absorption can either be non selective or selective. For example, charcoal and adsorption resin are less selective adsorbent. Solid phase coated with affinity molecule specific to the anti tumor agent can be used as adsorbent to selectively remove the anti tumor agent. These agents can also be neutralized by adding suitable neutralizer that can inactivate their activity instead of removing them. For example, spermine or protamine can be used to neutralize the cytotoxic effect of the alkylating agent. The treatment can be done either in a continuous flow fashion or intermittent flow fashion. For example, the blood is withdrawn continuously and then is added with CTC inactivating agent continuously and returned to the patient continuously. In another example, certain volume of blood/blood component is withdrawn and been treated for certain period of time with drug then return to the patient and then the next batch of blood/blood component is withdrawn for treatment. This will allow enough time for the CTC inactivating. It can also be the combination of continuous flow/intermittent flow. For example, the blood passing through the blood cell separator and adsorbent is done continuously but the CTC inactivating with anti tumor agent (adding agent and incubation), agent removing and blood component returning to the patient is done in batch. If the whole blood withdrawing and return is done in an intermittent flow fashion, single needle/catheter in the body can be used for both withdrawing and returning blood in a time slicing fashion by doing them in different time interval. Intermittent flow can be applied to the whole process or part of the process. The blood or blood components being treated with anti tumor cell agent can be in an intermittent flow (batch) fashion to enable they get desired time length of interacting with the agent (e.g. 5˜30 min for anti cancer drug to take effect, 5-10 min for photo dynamic treatment if it is used). In example 8, the blood flows to a chamber where suitable amount of anti tumor agent (e.g. to reach 20 times the IC50 of the drug concentration in the chamber) is added is paused when certain amount of blood (e.g. 200 ml)/blood components (e.g. 50 ml) is being treated inside with the added agent. After certain period of time (e.g. 20 min) the treatment is finished and the blood/blood component is released from this area and the next batch (for blood component may only have one batch) come into the chamber and the agent is added. The adding of CTC inactivating agent to blood or blood component and incubating of it with blood or blood component can be done in an intermittent flow fashion so the agent will have enough time to interact with the CTC. Other process such as blood withdrawn, blood returning and optionally blood separation can be either in a continuous flow fashion or intermittent flow fashion. Additional treatments such as CTC removal with affinity adsorbent can also be applied before the blood/blood component go back to the patient. After the whole treatment is complete, additional dialysis or blood purification can also be conducted for the patient to remove the drug from the blood.

A variation is that the drug is added to the blood but the blood returned to the patient directly without removing the added drug or the drug is injected to the patient's blood vessel directly without performing extracorporeal circulation, only after the completion of the treatment (e.g allowing drug stay in the blood for certain period of time), additional dialysis or blood purification is conducted to remove the added drug from the blood.

The whole CTC removing/inactivating treatment procedure of the current invention can be repeated several times to reach the desired effect. For example, it can be done at one day, three days, a week, a month and three months after the surgery or chemotherapy; or be performed based on the amount of the tumor cells in the blood. In many applications preferably the volume of the blood extracorporeally circulated should be more than the total blood volume of the patient in each treatment. More preferably, the volume is more than twice the total blood volume of the patient. In some embodiments each operation takes 2 hours at the blood flow rate of 100-200 ml/min. Many blood purification protocols and procedures can be readily available from reference. Varieties of strategy such as the micro particle detoxification system can also be adopted for the current inventions. The change of the amount of CTC before and after the blood purification can be used to evaluate the treatment effect and be used to determine if further blood purification is needed. If before and after the surgery and chemotherapy the amount of CTC is very low and does not increase, blood purification may not be needed. If the amount increase or is always high then blood purification is needed to reduce the CTC amount to a desired level. Although performing blood purification without other tumor treatment is also useful, it is preferred that a treatment (e.g. surgery, chemotherapy, radiation and etc.) capable of removing the source generating CTC is performed and then the blood purification (CTC removal/inactivation treatment) is performed to remove the residual CTC in the blood to prevent tumor recurrence and metastasis. In many applications preferably the first CTC removal/inactivation treatment is performed within one month after the tumor removal surgery. The CTC monitoring test can be performed, if the number is still high (e.g. >5 copies/ml), the treatment can be repeated (e.g. every 3 days or once a week) until the number is satisfactory. In one example, the first CTC removal/inactivation treatment is performed within a week after the surgery and then repeated once the next week, the next two week, the next month and the next two month, next 6 month and each 6 month later after. CTC monitoring test can be used to determine if more CTC removal/inactivation treatment is required. In another example, the first CTC removal/inactivation treatment is performed within a day after the surgery and then repeated once the next week, the next two week, the next month and the next two month. Further CTC monitoring test can be used to determine if more CTC removal/inactivation treatment is required. In a third example, the first CTC removal/inactivation treatment is performed right after the surgery and more CTC removal/inactivation treatment is performed every 3 days or every week until the CTC number is satisfactory. For patient receiving chemotherapy, the first CTC removal/inactivation treatment can be given within a week after the chemotherapy session end, the CTC count can be monitored frequently and more CTC removal/inactivation treatment can be given if the CTC count is high. The first CTC removal/inactivation treatment can be given within a week after the first dose of chemotherapy drug is given. More CTC removal/inactivation treatment can be repeated. For example, it is performed at one day, three days, one week, one month, and three months after the therapy and every 3 month later after. For patient receiving radiation therapy, photodynamic therapy, photon radiation therapy, laser therapy, microwave therapy, cryogenic therapy or heat therapy (e.g. hyperthermia therapy), the first CTC removal/inactivation treatment can be given within 1 week after the first therapy or within one week after the whole therapy end. More CTC removal/inactivation treatment can be repeated. For example, it is performed at one day, three days, a week, a month and three months after the therapy. The CTC count can be monitored frequently and more CTC removal/inactivation treatment can be given if the CTC count is high.

One can also use non-specific method to remove the tumor cells from blood/blood component (e.g. using active carbon filter, membrane differential filtration, cryofiltration, filters having suitable pore size, e.g. 8 um-12 um). The white blood cell filter can be used to remove the tumor cell when blood/blood component passes through. Cancer cells usually clump together for metastasis. Size based filtration can also be used to remove the clumped cancer cells. These cell clumps are bigger than blood cell size, therefore using a filter that can remove the clumped tumor cells but not the blood cells (such as filter with suitable pore size, e.g. 20 um) for blood purification after the surgery can also reduce the risk of metastasis. Similar blood purification methods can be found in many references as previously described.

In some embodiments, filtration or the like (e.g. geometrically-enhanced differential immune capture or geometrically-enhanced differential capture) is used to remove the CTC. Because the tumor cell is bigger than most of the blood cells, passing the blood or blood component (e.g. the white blood cell CTC mixture from the centrifugation type blood cell separator) through a filter having suitable pore size will remove the tumor cell but allow the normal blood cells going back to the patient, e.g. using filter membrane with pore size of 10 um, 15 um or 20 um. The filtrate can return to the patient or send to one or more other types of CTC removing/inactivating device/devices to eliminate the residual CTC before returning to the patient. The blocked CTC containing blood component can be discarded or treated with one or more other types of CTC removing/inactivating device/devices such as passing through a CTC affinity adsorbent particle filled blood purifier device or a CTC inactivating device and then the flow through are returned to the patient. Although white blood cells also have large size they can change the shape to pass the filter. Cancer cells usually clump together to have even larger size. Using a polycarbonate membrane having pore size of 8-15 um can efficiently remove most of the CTC but allow most blood cells pass through. Either single stage filtration or multiple stage filtrations can be performed. For example, several filter film can be coupled sequentially in a tandem fashion to perform the filtration. Using multiple stage filtrations allow the use of larger pore filter. Adding microsphere that can bind with tumor cell into the blood or blood component will provide a large size binding complex with CTC therefore will help the filter block the tumor cells. One can also use a small pore size filter or other methods (e.g. centrifuge, white blood cell separator) to remove both the white blood cell and tumor cell and then remove the tumor cell from the collected white blood cell and return the white blood cell and other blood components back to the patient. Examples of method to remove the tumor cell from white blood cell include using the immune magnetic particles or the solid phase CTC adsorbent described in the current inventions. One can also remove the white blood cell first then pass the tumor cell with other blood cells and plasma through a smaller pore size filter (e.g. 5 um, 10 um or 15 um) to remove the tumor cell but allow the red blood cell and platelet to pass and go back to the patient. There are many known ways and devices to remove the white blood cell such as using nylon fiber separator and other white blood cell separator. The separated white blood cell can be eluted from the separator and then send back to the patient. In an example, the patient having breast cancer is first treated with surgery to remove the tumor, next the patient is treated with leukapheresis using a blood cell separator. The collected leukocyte portion (200˜400 ml) is mixed with CTC adsorbent (e.g. 1 g of 1 um size polystyrene magnetic particle coated with EpCAM antibody or 5 ml 100 um size Sepharose 4B particle coated with EpCAM antibody) for 15 min, and then the CTC adsorbent is removed (e.g. using a magnet for magnetic particle or a filter of 60 um pore size for Sepharose 4B particle) and the cleaned leukocyte portion is returned to patient.

If the tumor cell affinity solid phase also has affinity to red blood cell/platelet but not to white blood cell, one can separate the red blood cell/platelet from the blood with other means then remove the tumor cell from the white blood cell with the said solid phase and send back the clean blood to the patient. If the tumor cell affinity solid phase also has affinity to white blood cell but not to red blood cell/platelet, one can separate the white blood cell from the whole blood with other means and then remove the tumor cell from the mixture of red blood cell/platelet with the said solid phase and send back the clean blood to the patient. The carbohydrate pattern on the tumor cell normally is different compared with the normal cell, one can use lectin specific to them to bind with it. For example, PHA (phaseolus vulgaris agglutinin) lectin can bind with gastric carcinoma cell and BSA lectin can bind with breast cancer cell. Coating lectin on the solid phase can also be used to remove CTC. The tumor cell surface has more native charge compared with normal cell because it has high density of surface sialic acid. The solid phase having positively charged groups or molecule such as chitosan, oligochitosan, poly glucosamine and other polymers having amine groups can be used to capture the CTC in the blood. It can also be combined with filtration. To prevent the red blood cell absorption, one can first remove the red blood cell (e.g. filtration, centrifuge) from the blood then perform the CTC removal, e.g. passing the non red blood cell containing blood components through a positively charged solid phase or filter to remove the CTC then send the normal blood cells back to the patient.

Because different cells have different density/size, one can also use centrifuge to remove the CTC. After centrifuge of the whole blood, the red blood cell is at the bottom and the white blood cell and CTC is on top of it. In order to get a better separation, high density particles (e.g. glass micro sphere, silica beads, magnetic bead) having affinity to CTC can be added to the blood so the resulting binding complex will have high density and will go to the bottom easily after centrifugation, therefore the upper part can be safely send back to the patient.

Adding microsphere that can bind with tumor cell into the extracorporeally circulating blood or blood component will provide a large size binding complex with CTC therefore will help the filter block the tumor cells. In example 9, to perform CTC removal, 300 um diameter Sephadex beads coated with antibody against CTC surface marker is added to the extracorporeally circulating blood and then the blood is passing through a filter having pore size of 200 um to remove the beads as well as beads bound CTC before the blood goes back to the patient. In one example, the process is done in a batch format. 300 ml of blood is withdraw from the patient and then mixed with 3 ml 300 um diameter Sephadex beads coated with EpCAM antibody in a chamber. The blood and Sephadex beads are incubated in the chamber with shaking for 5 min and then pass through a filter membrane having pore size of 200 um at the exit of the chamber to remove the beads and bound CTC. Next the filtrate (blood) is returned to the patient and another batch of 300 ml blood form the patient is sent to the chamber to mix with newly added 3 ml Sephadex beads to repeat the above process. The Sephadex beads after filtration can be removed from the chamber after each batch or after several batches. The operation can also be done in a continue flow fashion by performing blood withdrawing, mixing with beads, filtration and returning of blood continually. Other particle such as inorganic beads (e.g. silica beads), biodegradable beads and magnetic beads can also be used instead of Sephadex or the like solid phase support. Using magnetic particle allow the removal of the beads by the combination of filtration and magnetic separation. Unlike these beads used in micro particle detoxification system, the particle suitable for this method should be bigger than the CTC, e.g. >50 um, >100 um or >200 um to facilitate the filtration. The filter should allow most of the blood cell to pass though but retain the added particles. Furthermore, other shape of particle can also be used besides beads such as fibers, rod, cube and etc., as long as they can be removed with the filter used in the process.

Means that can kill the tumor cells can also be applied to the tumor cell removing device. For example, low temperature (e.g. −10 degree) or high temperature (e.g. 40˜60 degree) can be applied to the solid phase support (e.g. the column, filters, fibers and membrane) or the filter. Light (UV or visible light), microwave or radiation can also be applied. Because the tumor cell will stay longer/trap in the solid phase/filter, they will be cool/heat/light or radiation treated much longer time, by carefully control the intensity of the treatment, the tumor cells will be killed but most healthy blood cells will still be alive because they pass through the solid phase/filter quickly. So the flow speed, treatment intensity (e.g. temperature, light or radiation intensity) needs to be adjusted so that only the cells stay on the solid phase for a long time will be killed. So even if the tumor cells are released from the solid phase to the blood they still cannot cause new tumor growth. If photon is used to kill the CTC, photoactive agents such as phenothiazine dyes, methylene blue, vitamin B2, psoralen, photosensitizer agent used in photodynamic therapy can also be added to the blood to increase the CTC inactivating efficacy. These photoactive agents/photosensitizers can also be coupled with affinity ligand to the CTC to provide better selectivity.

One can also pass the blood through a cartridge that can selectively slow down the movement of the CTC so the CTC will receive long time of treatment in the cartridge to be killed. For example, the cartridge contains multiple layer of mesh (e.g. membrane or alignment of fiber or fiber textile) and the mesh size is much bigger than red blood cell but not too bigger than the CTC size (e.g. 20 um, 30 um, 50 um or 100 um). The mesh can also be coated with affinity ligand to tumor to capture the CTC. The cartridge can also contain multiple surfaces having relative obstacle structure alignment for the CTC, e.g. a lot of post on the surface with a distance of 80 um between each other.

In some preferred embodiments, the tumor treatment method of current inventions comprises the following step: 1) performing a tumor removal or inactivating treatment to the patient to remove the source generating CTC including surgery, chemotherapy, radiation, microwave, photon treatment, cooling or heating treatment; 2) performing blood purification to the patient to remove the CTC in the blood by extracorporeally circulating blood through solid phase having affinity to tumor cell or filter to remove the CTC from the blood and then return the blood to the patient.

In one example, patient having tumor is treated with whole blood purification to remove the circulating tumor cells right after the tumor removing surgery. The surgery can cause the release of tumor cells into blood therefore increase the risk of tumor metastasis. Some chemotherapy or radiation can also cause the release of tumor cells into blood. By removing them with blood purification after or during the surgery/chemotherapy/radiation treatment, the risk is decreased. Varieties of blood purification techniques can be used such as those described above. For example, one method is to use column immobilized with folic acid to selectively remove the tumor cells for blood in the blood pass through. Another example is to use none selective method such as active carbon absorption or white blood cell filter or membrane differential filtration to remove the tumor cells in the blood.

The solid phase support for blood purification (either whole blood or blood component purification) could be a column, a membrane, a fiber, a particle, or any other appropriate surface, which contains appropriate surface properties (including the surface of inside the porous structure) either for direct coupling of the affinity molecules or for coupling after modification or for surface derivatization/modification. If the solid support is porous, its inside can also be used to present the binding affinity molecules.

In some embodiments, the blood passes through a hollow fiber membrane, wherein affinity molecules for tumor cells are immobilized within a porous exterior portion of the membrane. Examples of affinity molecules are anti cytoketatins antibodies, EpCAM antibodies and any other antibodies against tumor cells (e.g. an antibody for prostate-specific membrane antigen for prostate cancer). The affinity molecules can be attached to a solid phase support matrix prior to being immobilized within the porous exterior portion of the membrane. One example of the solid matrix is sepharose or sephadex. Examples of the hollow fiber membrane can be found in U.S. Pat. No. 6,528,057 and U.S. Pat. No. 7,226,429. The blood purification protocol can also be readily adopted from these patents and other blood dialysis references.

In one example of the method of the present invention, blood is withdrawn from a patient and contacted with the ultrafiltration membrane having affinity molecules. In one embodiment, the blood or blood component is contacted with the adsorbent particle having affinity molecules specific for the tumor cells to remove them and returned to the patient. The treatment can be repeated periodically until a desired response has been achieved. For example, the treatment can be carried out for 2 hours every week. Thus, exemplary steps of the present invention are (a) contacting the body fluid with the affinity molecule immobilized to a surface under conditions that allow the formation of bound complexes of the affinity molecules and their respective target cells; (b) collecting unbound materials; and (c) reinfusing the unbound materials into the patient.

There are numerous methods for coupling a chemical to solid support. These methods are readily available from scientific journals, vendors that provide coupling reagents, or relevant websites. For example, chemicals containing a primary amine can be coupled to a solid support that is functionalized with a carboxyl group through the formation of amide bond; the formation of amide bond between the amine and carboxyl group is normally catalyzed with EDC [1-(3-dimethylaminopropyl)-3-ethylcarbodiimide hydrochloride] or other carbodiimide. The tumor cell binding chemicals may need to be appropriately modified or derivatized to introduce a functional group that can be used for coupling while at the same time the modification or derivatization does not inactivate the tumor cell binding activity. It is understood that the binding chemicals can be chemically or naturally coupled to another moiety that can be subsequently coupled to a solid support. In other examples, the tumor cell binding chemicals themselves could used to form the solid phase support.

These tumor cell binding chemicals are immobilized on solid support to remove tumor cells from blood. In example 10, coupling of folic acid to the particle can be performed as follows: 20 mg of particles having surface amine groups (e.g. the 0.2-0.5 mm diameter crosslinked dextran particle such as Sephadex beads or glass beads derivatized to have amine group) are washed three times with 0.1 M MES, pH 5.0 and again three times with deionized water. The particle wet cake is suspended in 0.5 mL of folic acid (Sigma-Aldrich) at 20 mg/mL in deionized water, followed by an addition of 0.5 mL of 20 mg/mL carbodiimide [1-Ethyl-3-(3-dimethyl-aminopropyl)-carbodiimide hydrochloride, EDC] in deionized water, which is prepared immediately before use. The pH is then adjusted to 7.5 with 0.1 M NaHCO₃ solution. The particles are rotated at room temperature for 2 hours. Another 10 mg of EDC and 10 mg of NHS (N-hydroxysuccinimide) are added to the mix, followed by an overnight rotation at room temperature. The particles are washed 3 times with 10 mM HEPES buffer, pH 7.5, 5 times with deionized water and then suspended in 1.0 mL of deionized water. The reagent is now ready to be packed in a column for use for tumor cell removal.

The current FDA approved circulating tumor cell detection method use one group of antibodies for all the tumors. They are the antibodies against the common markers for epithelial cells. Tumors are epithelial cells so it is a universal maker for tumor and therefore one group of antibodies for all. For example, EpCAM antibodies and anti cytokerantins antibodies can be coupled to the solid phase support in the blood purification device and therefore be used in the removal of circulating tumor cells from the patient's blood, preferably after the surgical removal of the tumor. In example 11, Cyanogen bromide (CNBr) activated agarose particle is used for direct coupling essentially according to Cuatrecasas, et al (Cuatracasas, Wilchek and Anfinsen. Proc Natl Acad Sci USA 61(2): 636-643, 1968). In brief, 1 ml of EpCAM antibody at a concentration of 10 mg/ml in 0.1M NaHCO₃ pH 9.5 is added to 1 ml CNBr activated agarose (around 100 um in diameter, e.g. CNBr-activated Sepharose 4B) and allowed to react overnight in the cold. When the reaction is complete, unreacted materials are aspirated and the antibody coupled agarose washed extensively with sterile cold PBS. The antibody coated agarose affinity matrix is then stored cold until ready for use.

In example 12, the affinity matrix is prepared by a modification of the method of Hermanson. Anti-cytokerantins antibodies dissolved to a final protein concentration of 10 mg/ml in 0.1M sodium borate pH 9.5 is added to aldehyde derivatized silica glass beads (200 um in diameter). The reaction is most efficient at alkaline pH but will go at pH 7-9 and is normally done at a 2-4 fold excess of protein over coupling sites. To this mixture is added 10 ul 5M NaCNBH3 in 1N NaOH per ml of coupling reaction and the mixture allowed to react for 2 hours at room temperature. At the end of the reaction, remaining unreacted aldehyde on the glass surfaces is capped with 20 ul 3M ethanolamine pH 9.5 per ml of reaction. After 15 minutes at room temperature, the reaction solution is decanted and the unbound proteins and reagents removed by washing extensively in PBS. The matrix is the stored in the refrigerator until ready for use.

In example 13, 30 ml particles coated with anti EpCAM antibody and 30 ml particles coated anti-cytokerantins antibodies prepared from the above examples are placed in a column shape vessel. 500 ml blood added with anticoagulant containing 1 million breast tumor cells is added to the vessel to pass the CTC removal particles inside. The blood passing though is collected at the exit of the vessel and more than 90% cancer cells can be removed.

In example 14, the tumor affinity solid phase (e.g. 100 ml the particles from the above example 11 or 12 or their equal mixture) is packed in a vessel (100 mm inner diameter and 200 mm inner height) to form the blood purifier (CTC removal device 29 in FIG. 9). One ends of the vessel 29 has blood inlet and another end has blood outlet to connect with the blood from artery and return blood to the vein as shown in FIG. 9. Filters with suitable pore size (smaller than the particle size but bigger than the blood cell, e.g. 80 um) are placed at the inlet and outlet of 29 to block the solid phase particle going out but allow the cells passing freely. The patient first undergoes a tumor (e.g. lung tumor or skin tumor or breast tumor) removal surgery and after two days the blood purification using the above purifier is performed to remove the CTC. First the extracorporeally circulating path is established, the blood comes out from the artery of the patient goes into the blood inlet of the blood purifier with the aid of blood pump 28 and pass through the solid adsorbent inside 29 and then goes out from the blood outlet and infuse back to the vein of the patient. The blood flow rate is 100 ml/min and the operation last for 2 hours.

In example 15, the hollow fiber blood dialyzer used for blood dialysis is used as vessel for the solid phase CTC adsorbent of 200 um size. The hollow fiber has an inner diameter of 300 um. 30 ml solid phase adsorbent particle described in the above examples is filled inside the hollow fiber. The blood inlet and outlet is sealed with filter membrane having pore size of 100 um. The patient first undergoes a radiation therapy, and after one day the blood purification using the above purifier is performed to remove the CTC. First the extracorporeally circulating path is established with anticoagulation treatment, the blood comes out from the artery of the patient goes into the blood inlet of the blood purifier and pass through the solid adsorbent and then goes out from the blood outlet and infuse back to the vein of the patient. The blood flow rate is 100˜200 ml/min and the operation last for 2 hours. The blood purification to remove the immune suppression factor can also be performed at the same time by filling the suitable solid adsorbent in the vessel too (either inside the hollow fiber or outside the hollow fiber) at the same time. Alternatively, the whole blood first go through a leukapheresis device such as a blood cell separator and only the white blood cell portion containing the CTC passes though the blood purifier and then send back to the patient. The other blood components (e.g. plasma, red blood cell and platelet) are sent back to the patient directly without passing the blood purifier.

In example 16, the hollow fiber or the filtration membrane itself in the CTC removal device can be used as solid phase to capture CTC instead of filling additional CTC capture solid phase particle. The tumor cell affinity molecule can be immobilized on their surface. For example, the EpCAM antibody is to be coupled directly to polysulfone hollow-fibers in situ in a plasma separator cartridge or a blood dialysis cartridge for kidney failure. To accomplish this, the cartridge is first exposed to a solution of 4% human serum albumin (HSA) reacted overnight at 4 degree. The adsorbed HSA is then cross-linked with glutaraldehyde. Excess glutaraldehyde is then briefly washed out with water. The cartridge is then filled with cyanoborohydride coupling buffer containing 2-3 mg/ml of the antibody and reacted overnight at 4 degree. At the end of the reaction, excess antibody is washed off and the remaining unreacted aldehyde reacted with ethanolamine. After final washing in sterile PBS, the cartridge was dried in sterile air, packaged and sterilized using gamma-irradiation (25-40 kGy) and stored in a cool, dark area until ready for use.

In example 17, the above CTC removal blood purifiers are placed in a microwave generator during the blood purification during the extracorporeally circulating treatment. The power of the microwave generator is adjusted to keep the purifier inside at temperature of 46˜50 degree. Therefore the tumor cell trapped will be killed.

A small amount of blood (e.g. 1˜100 ml) can be withdrawn from the patient and be tested with a CTC removing/inactivating method in vitro in a small scale to predict its CTC removing/inactivating efficacy for the patient in full scale extracorporeally blood circulating treatment using this method. If the efficacy and safety is satisfactory, this method will be used to treat the patient in full scale extracorporeally blood circulating. Preferably the small blood volume in vitro test should provide a close mimic in a small scale to full scale extracorporeally blood circulating treatment. Because only a small amount of blood is tested, the size of the device can be miniaturized, the amount of the reagents used can be reduced and the time can be shortened compared with those used for whole blood volume extracorporeally blood circulating treatment. The procedures can also be modified to fit the in vitro test format. The relationship between the efficacy from the small volume blood in vitro test, the efficacy from the large volume blood (e.g. 1 L-4 L) in vitro test and the efficacy in extracorporeally blood circulating treatment to the patient (real treatment) can be first determined experimentally. The small volume blood in vitro test in which the efficacy has good correlations with the efficacy shown in large volume blood in vitro test/extracorporeally blood circulating treatment to the patient is preferred to be used to predict the efficacy of the extracorporeally blood circulating treatment. In example 18, a 0.5 cm diameter small column filled with 2 ml of CTC adsorbent from example 11 is used as in vitro test that can be used predict the CTC removal efficacy of the method in example 14 using a cartridge containing 100 ml CTC adsorbent from example 11. To perform the in vitro test, first 30 ml blood is withdrawn from the patient. 15 ml blood sample is used for test using the small column and another 15 ml blood does not (intact as control). The in vitro test is performed by circulating the 15 ml blood through the column for 20 min at 1 ml/min flow rate. Then the CTC numbers in the two samples are counted and reduction rate of CTC from in vitro test can be calculated readily. After the 30 ml blood withdrawn, the patient receive the whole volume blood extracorporeally blood circulating treatment as described in example 14 using a cartridge containing 100 ml CTC adsorbent from example 11. After the treatment, the CTC is also counted and the reduction rate is calculated. Then relationship (e.g. a mathematical model or formula) between the efficacy of the in vitro test and the efficacy of the real patient treatment can be determined from the two reduction rates. The above procedure can be performed to multiple patients and the resulting data can be used to provide a relationship more suitable for predicting the real treatment result using in vitro test for an untreated patient. For example, if the resulting relationship indicate a CTC reduction rate of 60% from in vitro test correlate a 40% reduction rate in real treatment using a specific method and a patient showed 60% reduction rate in from vitro test using his blood, the predicted real treatment CTC reduction rate would be 40% using this method. A physician can use this predicted treatment efficacy to determine if the real treatment using this method should be performed to this patient. It is known that different real treatment methods require different in vitro tests and the resulting relationship may not be used interchangeable. The parameters in the in vitro test can also be modified as long as it can still provide good prediction for the real treatment (provide good correlation between the two CTC reduction rate). For example, one can use different CTC adsorbent amount (e.g. 1 ml), different size column (e.g. 0.2 cm diameter), different blood volume (e.g. 10 ml), passing the blood through the column only once at a flow rate of 2 ml/min instead of circulating and etc as long as good correlation between the two CTC reduction rate in multiple patients still exist. The optimal parameter value can be determined experimentally by varying the parameters in the in vitro test for the best prediction capability.

In example 19, a patient receives a whole volume blood extracorporeally blood circulating treatment as described in example 14 using a cartridge containing 200 g CTC adsorbent from example 12. The cartridge is 5 cm in diameter and 20 cm in height. A 0.5 cm diameter ( 1/100 of the flow through area of the real treatment) small column filled with 2 g of CTC adsorbent ( 1/100 of that used in real treatment) from example 12 is used as in vitro test that can be used to predict the CTC removal efficacy of real treatment.

To perform the in vitro test, 40 ml blood is withdrawn from the patient before the real treatment. 20 ml blood sample is used for test using the small column and another 20 ml blood does not (intact as control). The in vitro test is performed by passing the 20 ml blood through the column at 1 ml/min flow rate ( 1/100 of the flow rate of the real test) and collecting the filtrate. Then the CTC numbers in the filtrate and the control samples are counted and reduction rate of CTC from in vitro test can be calculated readily. At the same time the patient also receive the whole volume blood extracorporeally blood circulating treatment. After the treatment, the CTC is also counted and the reduction rate is calculated. Then relationship (e.g. a mathematical model or formula) between the efficacy of the in vitro test and the efficacy of the real patient treatment can be determined from the two reduction rates. The above procedure can be performed to multiple patients and the resulting data can be used to provide a relationship more suitable for predicting the real treatment using in vitro test for a patient. For example, a curve can be drawn based on the data points where each point represent a patient in which x is the reduction rate from the in vitro test and y is the reduction rate from real treatment for this patient (other parameter can also be included in the curve such as the type of cancer as well as original CTC count in the patient, which can be used as Z axis value or making clusters). In order to predict a new patient's real treatment efficacy, his blood can be drawn for the in vitro test described above. The resulting CTC reduction rate can be used as x to get the corresponding y value using the curve. The resulting y value is the predicted real treatment CTC reduction efficacy.

Alternatively, large volume blood (e.g. 1 L-4 L) in vitro test can be used instead of the whole volume extracorporeally blood circulating treatment in human to establish the prediction relationship for the real treatment in the methods described above. It can also be used to optimize the small volume blood in vitro test for better correlation with real treatment. Human body contains 3˜4 L blood. Using a large volume blood sample instead of extracorporeally blood circulating can simplify the procedures and provide a close mimic to that in the real human. A reservoir containing a large volume blood having a blood outlet and blood inlet can be used as a human dummy model in the extracorporeally blood circulating treatment.

In example 20, a 0.5 cm diameter small column filled with 2 ml of CTC adsorbent from example 11 is used as in vitro test that can be used predict the CTC removal efficacy of the method in example 14 using a cartridge containing 100 ml CTC adsorbent from example 11. To perform the in vitro test, 30 ml blood is withdrawn from the patient. 15 ml blood sample is used for test using the small column and another 15 ml blood does not (intact as control). The in vitro test is performed by circulating the 15 ml blood through the column for 20 min at 1 ml/min flow rate. Then the CTC numbers in the two samples are counted and reduction rate of CTC from in vitro test can be calculated readily. At the same time the 4 L of anticoagulant treated human blood spiked with 1 million lung cancer cell is placed in a container which has a blood inlet and a blood outlet. The blood outlet is connected with a blood pump to drive the blood pass through a cartridge used for example 14, which contains 100 ml CTC adsorbent from example 11. The blood exit the cartridge then goes back to the container through its blood inlet. The flow rate is 100 ml/min and the process last for 2 h. After the treatment, the CTC in the container is also counted and the reduction rate is calculated. Then relationship (e.g. a mathematical model or formula) between the efficacy of the in vitro test in small blood volume and the efficacy of the in vitro test in large blood volume can be determined from the two reduction rates. The above procedure can be performed to multiple large volume blood samples which spiked with different amount/type of cancer cells. The resulting data can be used to provide a relationship more suitable for predicting the real treatment result using small volume blood vitro test for an untreated patient. For example, if the resulting relationship indicate a CTC reduction rate of 60% from small volume in vitro test correlate a 40% reduction rate in large volume test using certain method when the lung CTC count is 50/ml and a lung cancer patient having a CTC count of 50/ml showed 60% reduction rate from in vitro test for his blood, the predicted real treatment CTC reduction rate would be 40% using this method. A physician can use this predicted treatment efficacy to determine if the real treatment using this method should be performed to this patient. The parameters in the small amount blood in vitro test can also be modified and their correlation with the large volume test (between the two CTC reduction rates) is compared. For example, one can use different CTC adsorbent amount (e.g. 1 g), different size column (e.g. 0.2 cm diameter), different blood volume (e.g. 10 ml), pass the blood through the column only once at a flow rate of 2 ml/min instead of circulating and etc. Therefore, the optimal parameter value can be obtained experimentally by varying the parameters in the small volume in vitro test and selecting those having the best correlation/prediction capability for large volume blood in vitro test.

Using small volume blood in vitro test is not limited to CTC removal applications as described above. The same strategy can also be used for virus/pathogen removal/inactivation using extracorporeally circulating blood methods described in the current inventions or those used by others. In example 21, 30 ml of blood is withdrawn from a patient having HCV infection. The blood is low speed centrifuged and the plasma part is divided into two equal portions. One part is intact and another part is irradiated with UV light of 253 nm at the intensity of 60 uW/cm² for 30×6=180 seconds. This condition is to mimic the UV treatment in example 3. In example 3, the flow rate is 200 ml/min. Since human average blood volume is 4000 ml, extracorporeally circulating 2 h will circulate the blood 120×200 ml, which is 6 times the total blood volume. In another word, the plasma is irradiated 6 times during the treatment (each times 30 s). Therefore, UV light of 253 nm at the intensity of 60 uW/cm² for 180 seconds for the in vitro test will ensure the small volume blood sample receive the same amount of UV irradiation as that in the real patient treatment in example 3. Next the HCV inactivation rate is determined by testing the viability of HVC virus in the two plasma part (e.g. using culture method). The HCV inactivation rate in this in vitro test is reported to the physician, who can use this information to decide if the patient should be treated with the extracorporeally blood circulating method described in example 3. For example, if significant amount of HCV is inactivated (e.g. >60%), then the patient is sensitive to this treatment and this treatment is recommended. In example 3, additional cartridge filled with HCV adsorbent can be used in to further clean the HCV in the plasma. Similar to those described examples 18˜20, a small column filled with the HCV adsorbent used in example 3 can also be used as in vitro test to predict the HCV removal efficacy of the cartridge used in example 3. For example, 30 ml of blood is withdrawn from a patient having HCV infection. The blood is low speed centrifuged and the plasma part is divided into two equal portions. One part is intact and another part pass through a 0.5 cm diameter small column filled with 1 ml of HCV adsorbent used in example 3 at the flow rate of 1 ml/min. The HCV count is tested in the intact plasma and the treated plasma (e.g. using PCR or ELISA) and the reduction rate is determined. If significant amount of HCV is removed (e.g. >60%), then the patient is sensitive to the cartridge in example 3 and it can be used for the patient either in combination with the UV treatment or alone without the UV treatment. If only small amount of HCV is removed (e.g. <30%), additional means such as a cartridge can remove the lipoprotein-HCV complex or the double filtration method can be used for the patient. Furthermore, similar to those described in examples 18˜20, multiple patients can be tested using the small amount blood in vitro test and receive the extracorporeally blood circulating HCV removal treatment in example 3 (using the HCV removal cartridge but no UV treatment). The HCV removal rates are determined in the in vitro test and the real treatment. Their relationship (e.g. a curve) is determined and can be used to predict the HCV removal efficacy of the real HCV removal treatment for new patient from his in vitro blood test.

Using small volume blood in vitro test is not limited to CTC/pathogen removal/inactivation applications and as described above. The same strategy can also be used for other blood purification technologies using extracorporeally circulating blood methods. Suitable blood purification technologies include but not limited to hemodialysis, hemofiltration, plasmapheresis, apheresis, hemoperfusion, hemopurification, plasmapheresis, blood perfusion, plasma exchange and immune absorption. Similar to those for CTC/pathogen removal/inactivation applications, the small volume blood in vitro test that mimic the specific blood purification technology for the patient in a small scale can be used to predict the efficacy of the blood purification technology used in the patient. Furthermore, the safety (side effect) can also be predicted by the small volume blood in vitro test in which the factor (e.g. change of bradykinin level, hemolysis, reduction of beneficial component such as HDL and etc.) causing the safety issue/side effect is also measured and used to predict the factor in the real treatment for the patient. For example, the LIPOSORBER system uses dextran sulphate cellulose as adsorbent to remove the low-density lipoprotein cholesterol (LDLC) in the patient's plasma. A small column filled with dextran sulphate cellulose can be used to test a small amount of patient's plasma to predict the efficacy of LIPOSORBER system for the patient. For example, 10 ml of plasma from a patient having high LDLC pass through a 0.5 cm diameter small column filled with 1 ml of dextran sulphate cellulose used in LIPOSORBER at the flow rate of 1 ml/min and the LDLC level in the plasma sample before and after the column is measured. The high-density lipoprotein cholesterol (HDLC, good to health) level in the plasma sample before and after the column is also measured. LIPOSORBER system may also remove considerably amount of HDLC in some patients, which raise potential safety concern. The patient is then treated with LIPOSORBER system and the LDLC and HDLC before and after treatment is also measured. By repeating this process in multiple patients, a relationship model between the result from the in vitro test and the LIPOSORBER treatment can be determined, which can be used to predict the efficacy (LDLC reduction rate) and the safety (HDLC reduction rate, the lower the better) of the LIPOSORBER treatment for a new patient by using the result from testing his plasma sample with the small volume in vitro test described above. A physician can use the predicted efficacy and safety to decide if the LIPOSORBER treatment should be used for this patient or not. In another example, a small amount blood in vitro test is used to predict the effect of heparin induced extracorporeal lipoprotein precipitation (HELP) to a patient. The in vitro test is performed as following: 10 ml of plasma from a patient having high LDLC is mixed with the same heparin buffer at same ratio to plasma as those used in HELP for the patient. Next the precipitation is removed use the same method as that in HELP. The LDLC and HDLC level in the plasma sample before and after the in vitro test is measured. If the reduction rate of LDLC and HDLC level in the plasma sample is satisfactory, HELP can be performed to the patient. Furthermore, the above in vitro test can be performed to multiple patients before they have the HELP treatment. The change of the LDLC and HDLC level in the in vitro test and the HELP treatment is used to produce a prediction model based on the relationship between those in the in vitro test and those in the HELP. When the new patient come, an in vitro test can be performed using his plasma sample and the result is inputted into the prediction model to produce a prediction for the efficacy and safety of HELP for him. There are many different LDLC methods/devices available now, e.g. direct adsorption of lipoprotein from whole blood (DALI), HELP, LIPOSORBER, Immunoadsorption system with special antilipoprotein(a) column, membrane different filtration (MDF), dextran sulfate cellulose adsorption (DSCA) and etc. One can also build corresponding in vitro tests using small amount of blood for each of these methods. When a patient comes, all or some of these tests can be performed to his blood sample and the test shows the best result is determined (e.g. the best LDLC removal efficacy or the best efficacy/safety index). The corresponding treatment method can be recommended to the patient. In some case the prediction model need not to be built if these in vitro tests use similar conditions (e.g. similar blood volume, flow rate and etc.). In another example, an in vitro test using a small amount of blood is developed for to predict the efficacy of Immunosorba (Fresenius) to treat systemic lupus erythematosus (SLE) patient by removing the auto antibody (e.g. anti-ds-DNA antibodies). A small amount of blood can be withdrawn from the patient to perform the in vitro test (e.g. passing it through a small column filled with the same Protein A coupled solid phase in a small amount and measuring the auto antibody reduction rate) and the result is used to determine if Immunosorba should be used for the patient.

Before removing the circulating tumor cells from the blood and/or inactivating the circulating tumor cells with extracorporeally circulating blood, one can withdraw a small amount of blood (e.g. 10˜50 ml) from the patient and test it with a in vitro test mimic the method to be used for its in vitro efficacy of removing/inactivating the CTC. Only if significant amount of CTC in the blood sample is removed or inactivated the full scale treatment using this method with extracorporeally circulating blood will be used to the patient. Otherwise a different method will be tested with a small amount of blood to find out the best method to remove/inactivate the CTC for the patient. Alternatively a small amount of blood is withdrawn and divided to several portion, each is treated with a different CTC removal/inactivating method in vitro and the results are compared, the method shows the best efficacy will be used as the treatment for the patient if they have similar safety profile. If they have different safety profile, the method having high efficacy yet low side effect will be used. Because only a small amount of blood (e.g. 1˜200 ml) is tested instead of liters of blood during extracorporeally circulating blood, a smaller scale of device/reagent and a shorter time can also be used instead. Part or the whole procedure of the method to be used to the patient will be performed to the blood sample to predict its efficacy during extracorporeally circulating blood. If no significant amount of CTC (e.g. <15%) is reduced/inactivated using this method when testing this small amount blood sample, this method will not be used. The method will be used to the patient only when significant amount of CTC in the blood sample is removed or inactivated (e.g. in some cases, >25% is required; in another cases, >50% is required) for the small amount of blood sample. For example, 20 ml blood can be withdrawn from the patient and a smaller size cartridge containing a small amount of CTC adsorbent can be used in vitro for this blood sample to predict if a regular size cartridge with more CTC adsorbent should be used for whole volume blood extracorporeally circulating treatment. The size of the cartridge and amount of CTC adsorbent for the test can be reduced accordingly based on the difference between the volume of the blood sample and the blood volume of the patient. For example, one can use a small column filled with 1˜2 g of CTC adsorbent for 20 ml blood in vitro test if the cartridge for the patient treatment containing 100 g CTC adsorbent. In one example, during the in vitro test, 30 ml blood is withdrawn from the patient. 15 ml blood sample passes though the small column filled with 1 g CTC adsorbent and another 15 ml blood does not. Then the CTC in the two samples are checked. If more than 50% of the CTC in the blood sample treated with column is removed, the corresponding treatment cartridge can be used for the patient. It is understood the structure of the device, the parameter and the procedure for the in vitro test need not to be exactly identical to that used to treat the patient, e.g. the size, time, flow rate can be adjusted to fit the in vitro test format as long as the in vitro test can give a prediction of the efficacy of the treatment for the patient. The size, density, surface marker of the CTC varies so sometimes one CTC removal method suitable for one patient may not be suitable for another patient. A successful result in a small scale in vitro test will ensure the efficacy of extracorporeal circulating treatment. In another example, 20 ml of blood sample from the patient passes through a size reduced filter type device or simply the filters (smaller surface area but same pore size) used in the device, if more than 70% CTC is removed, the filter type device will be used to the patient. In another example a small amount of blood is tested in vitro using a centrifugation based blood cell separator or a similar centrifugation device to see if CTC can be successfully separated from most of the other blood cells before this method is used for the patient. Similarly, the CTC inactivating method such as those using drug or exogenous material or physical means as previously described can also be tested in vitro with a small amount of blood sample from the patient before certain method is used for this patient. The combination of several methods/devices (size reduced if necessary) can also be tested in vitro using small amount of blood sample from the patient and if the overall CTC removing/inactivating efficacy is satisfactory, the combination will be used to treat the patient.

Furthermore, the result of the CTC removal/inactivating treatment can also be used for guiding further chemotherapy or other type of treatment (e.g. radiation therapy). If after a few CTC removal/inactivating treatment the high CTC number in the blood goes back again; residual tumor sites in the patient may be present which is not totally removed by the previous treatment or new tumor need to be discovered. New chemotherapy or other type of treatment (e.g. radiation therapy, surgery to remove the nearby tissue, lymph node, or newly discovered tumor) may need to be conducted. The CTC collected from the blood can be cultured with anticancer drugs to select the effective drug to treat the tumor for the patient by checking if the drug can inactivate the tumor cell during culture.

Therefore, the method to prevent tumor metastasis and tumor recurrence in the current invention comprises three steps 1) removing the tumor or treating the tumor with therapeutical means such as surgery, chemotherapy, radiation therapy, photodynamic therapy, photon radiation therapy, laser therapy, microwave therapy, cryogenic therapy, heat therapy or combinations of them; next 2) testing a small blood sample from the patient in vitro to predict the efficacy of one or more circulating tumor cells removal/inactivating methods 3) selecting the suitable method and using it to remove the circulating tumor cells from the blood and/or inactivate the circulating tumor cells by extracorporeally circulating blood.

All patents and publications mentioned in this specification are indicative of the level of those skilled in the art to which the invention pertains. All patents and publications are herein incorporated by reference to the same extent as if each individual publication was specifically and individually indicated to be incorporated by reference. The inventions described above involve many well known chemistry, instruments, methods and skills. A skilled person can easily find the knowledge from text books such as the chemistry textbooks, scientific journal papers and other well known reference sources.

1. A method to remove the pathogens in the blood of a patient, comprising:
separating the plasma from the extracorporeally circulating blood;
inactivating the pathogen in the plasma with physical means, and;
returning the plasma to the extracorporeally circulating blood or to directly to the patient.

2. The method according to claim 1, wherein the physical means is selected from UV radiation, microwave radiation and heating.

3. A method to treat cancer, comprising:
removing the tumor or treating the tumor with therapeutical means, and;
removing/inactivating the circulating tumor cells in the extracorporeally circulating blood.

4. The method according to claim 3, wherein the therapeutical means is selected from surgery, chemotherapy, radiation therapy, photodynamic therapy, photon radiation therapy, laser therapy, microwave therapy, cryogenic therapy, heat therapy or combinations of them.

5. The method according to claim 3, wherein the circulating tumor cells are removed by passing the extracorporeally circulating blood through a circulating tumor cell removal device.

6. The method according to claim 5, wherein the circulating tumor cells in the circulating tumor cell removal device is counted.

7. A method to treat autoimmune disease caused by the production of disease causing antibody, comprising:
removing the said antibody in the extracorporeally circulating blood, and then;
inactivating the cells producing the said antibody.

8. The method according to claim 7, wherein said antibody is removed by passing the extracorporeally circulating blood through an antibody removal device.

9. The method according to claim 7, wherein said cells is inactivated by antigen-cell inactivating agent conjugate that can specifically bind with the said antibody.
